(19) United States
(12) Patent Application Publication
(10) Pub. No.: US 2026/0146197 A1
(43) Pub. Date: May 28, 2026

(54) USE OF PEROXY GLUCONIC ACID IN VARIOUS TREATMENT FLUIDS

(71) Applicant: Secure Specialty Chemicals Corp., Calgary (CA)

(72) Inventors: Taryn FORSTER, Okotoks (CA); Douglas BRUCE, Calgary (CA)

(21) Appl. No.: 19/396,525

(22) Filed: Nov. 21, 2025

Related U.S. Application Data
(60) Provisional application No. 63/723,972, filed on Nov. 22, 2024.

(51) Int. Cl. C09K 8/52; A01N 25/02; A01N 59/00; A01P 1/00; A61L 2/18; C02F 1/50; C02F 5/10; C09K 8/60; C11D 3/20; C11D 3/39

(52) U.S. Cl. CPC C09K 8/52; A01N 25/02; A01N 59/00; A01P 1/00; A61L 2/186; C02F 1/50; C02F 5/10; C09K 8/605; C11D 3/2086; C11D 3/3942; C02F 2303/04

(57) ABSTRACT

The present disclosure generally relates to compositions containing peroxy gluconic acid, treatment fluids containing such compositions and methods of using the treatment fluids during various oil- and gas-field operations, water treatment, paper and pulp processing, and general disinfection.

### CROSS REFERENCE TO RELATED APPLICATIONS

This patent application claims the benefit of U.S. Provisional Application No. 63/723,972, titled “USE OF PEROXY GLUCONIC ACID IN VARIOUS TREATMENT FLUIDS”, filed Nov. 22, 2024, the contents of which are expressly incorporated by reference herein.

### TECHNICAL FIELD

The present disclosure generally relates to compositions containing oxidation products and/or oxidative derivatives of carbohydrates, such as saccharides, glucose, or the like, and methods of using the composition in treatment fluids for diverse applications, such as oil- and gas-field operations.

### BACKGROUND

Treatment or cleaning fluids can be used in various industries and operations for a wide range of purposes. These fluids, hereinafter referred to as “treatment fluids,” can be used in applications, such as water treatment, paper and pulp processing, oil- and gas-field operations, and cleaning and disinfection across residential, commercial, industrial, and medicinal settings. Common uses for treatment fluids include improving the performance of equipment, maintaining system stability, preventing deposits, enhancing material processing, and removing contaminants or residues.

In oil- and gas-field operations, treatment fluids are commonly used for a variety of purposes, including lubrication and cooling of drill bit cutting surfaces while drilling generally or drilling-in (i.e., drilling in a targeted petroliferous formation), transportation of cuttings (pieces of formation dislodged by the cutting action of the teeth on a drill bit) to the surface, controlling formation fluid pressure to prevent blowouts, maintaining well stability, suspending solids in the well, minimizing fluid loss into and stabilizing the formation through which the well is being drilled, minimizing fluid loss into the formation after the well has been drilled and during completion operations such as, for example, perforating the well, replacing a tool, attaching a screen to the end of the production tubular, gravel-packing the well, or fracturing the formation in the vicinity of the well, displacing the fluid within the well with another fluid, cleaning the well, testing the well. Treatment fluids can also be used for emplacing a packer and packer fluid, abandoning the well or preparing the well for abandonment, and otherwise treating the well, the subterranean formation, or any surface or downhole equipment used in connection therewith.

In the context of water treatment, treatment fluids can help maintain water quality and ensure efficient operation of systems. Polymers like polyacrylamides are often used for flocculation, where suspended particles are aggregated to facilitate sedimentation or filtration, viscosity enhancement, or stabilization, but can cause operational inefficiencies if deposited. However, excessive use or improper handling of these polymers can lead to deposits or fouling in water systems, including pipes, filters, and storage tanks. Such deposits can reduce system efficiency, increase maintenance costs, and compromise water quality. Treatment fluids should target the removal or degradation of polymers like polyacrylamides. Treatment fluids in water systems may also address issues, such as scaling caused by inorganic mineral precipitation (e.g., sulfates, carbonates) and microbial growth that can lead to biofouling. Effective treatment fluids in this context may inhibit scale formation and exhibit biocidal properties to control microbial contamination in addition to degrading polymeric residues.

In paper and pulp processing, treatment fluids may be used to help with controlling process efficiency and ensuring product quality, for example, by controlling polymer buildup. The paper manufacturing process may involve the use of polymers such as polyacrylamides for controlling the rheology of pulp suspensions, improving drainage on forming fabrics, and enhancing the retention of fines and fillers. However, over time, these polymers can accumulate on machinery surfaces, forming deposits that reduce equipment performance, increase downtime, and degrade the quality of the paper. Furthermore, the presence of organic or inorganic residues in the system can disrupt critical processes such as bleaching or sizing, leading to inconsistent product properties. Treatment fluids used in paper and pulp operations should be capable of effectively degrading polymeric deposits, removing organic and inorganic contaminants, and ensuring the efficient functioning of machinery and processes.

In cleaning and disinfection applications across residential, commercial, industrial, and medicinal settings, treatment fluids can be used to clean and sanitize surfaces, equipment, and environments. These fluids often exhibit biocidal activity to eliminate microorganisms such as bacteria, viruses, and fungi while removing residues that can harbor these microbes. For example, in residential or commercial cleaning, treatment fluids may be applied to surfaces prone to microbial contamination or soiling, such as countertops, floors, and bathrooms. In industrial settings, treatment fluids can be used to clean manufacturing equipment, maintain sterile environments, and prevent contamination in production processes. In medicinal applications, such as hospitals or pharmaceutical manufacturing facilities, treatment fluids may be required to meet stringent standards for biocidal efficacy and residue removal to ensure patient safety and product integrity. Effective treatment fluids in these contexts should be non-toxic, environmentally friendly, and capable of removing a wide range of contaminants, including organic residues, mineral deposits, and microbial biofilms.

Treatment designs typically mandate such fluids exhibit a certain level of viscosity, stability, or other properties. Polymers, such as polyacrylamides, are often used to provide the requisite viscosity, stabilization, or other properties. In oil- and gas-field operations, polyacrylamides are often used to form viscous gels to prevent fluid loss into the formation. In treatment fluids used for drilling, such polymers serve to suspend solids and assist in floating debris out of the wellbore. In treatment fluids used for stimulation, such polymers are used to increase the capability of proppant transport into the fracture during the fracturing operation and to also reduce friction, control fluid loss and control fracture geometry. Polyacrylamides can be used in water treatment to aggregate fine particles, enhance sedimentation, or stabilize flocculated solids. In paper processing, polyacrylamides can be used to improve pulp drainage and retention. In cleaning fluids, polyacrylamides can be used to suspend debris and enhance their removal from surfaces. In water treatment or paper and pulp processing, polyacrylamides may be used to enhance viscosity or flocculation but may lead to fouling or deposits that disrupt system performance. In water treatment systems, for instance, polyacrylamides can contribute to fouling if not properly degraded, while in paper and pulp processing, they may accumulate on machine components, requiring frequent maintenance. Similarly, in cleaning and disinfection applications, polymer residues may interfere with surface sanitization or lead to undesirable buildup over time. If left in the system, the polymers can deposit and also crosslink with metal ions to cause damage to formations, materials, or equipment. Therefore, effective means of degrading the polymer and polymer-metal complexes is desired in order to undo these effects (e.g., polymer's viscosity-modifying effects within the treatment fluid), minimize fouling (e.g., minimize or remove any deposits that may form), and optimize performance, whether in oil- and gas-field applications, water treatment systems, paper and pulp processing, or disinfection.

Moreover, treatment fluids (e.g., water that is produced during the above operations) can become saturated with inorganic minerals. Any change in pressure or temperature can alter the equilibrium between the water and the saturated minerals. With this disturbance in equilibrium, it is possible for the inorganic mineral components to form a precipitate, such as in the form of sulfates (e.g. barium sulfate, strontium sulfate, calcium sulfate), chlorides (e.g. barium chloride, strontium chloride, calcium chloride), carbonates, iron sulfides, etc., leading to deposits on pipes, vessels, surfaces, and various process equipment, thereby disrupting system operation, reducing operational efficiency and flow, and increasing maintenance costs. The issue may be prevalent not only in oil- and gas-field operations but also in water treatment, paper and pulp processing, and general cleaning or disinfection applications, where deposits or fouling can interfere with the intended action of the fluid. For instance, in oil and gas production, precipitates may disrupt the free flow of hydrocarbons, while in water treatment, they may clog systems or reduce treatment efficacy. For example, in water treatment and cleaning systems, precipitates can form deposits on equipment, pipes, or surfaces. In paper and pulp processing, mineral deposits can accumulate in processing lines or on machine components, affecting the quality of the paper and leading to production inefficiencies. In disinfection applications, mineral scaling on surfaces can interfere with the efficacy of cleaning agents and provide a substrate for microbial growth. Treatment fluids in these applications should therefore be designed to inhibit mineral precipitation, dissolve existing deposits, and maintain the integrity of equipment and surfaces.

Accordingly, it would be desirable to develop improved treatment fluids containing an agent that has low toxicity and is capable of at least one of: reversing the viscosity-modifying effects of polymer additives; degrading and/or removing polymer additive deposits or polymer-metal complexes; inhibiting the formation of and/or removing inorganic mineral precipitates; providing biocidal activity at low concentrations; and removing deposits (organic, inorganic or mixtures thereof) from surfaces of equipment or materials across various applications, such as oil- and gas-field operations, water treatment, paper and pulp processing, and disinfection.

### SUMMARY

According to an aspect of the present disclosure, a method for treating a substrate is provided. The method includes providing a treatment fluid comprising gluconic acid and hydrogen peroxide. The method also includes contacting the substrate with the treatment fluid for an effective amount of time to at least one of remove a contaminant, reduce a microbial population, or prevent formation of a polymer-polyvalent metal ion complex deposit.

According to other aspects of the present disclosure, the method may include one or more of the following features. The treatment fluid may further comprise citric acid. The treatment fluid may comprise from about 1 ppm to about 40 wt % of the gluconic acid and hydrogen peroxide combined, based on a total weight of the treatment fluid. The treatment fluid may further comprise methanol in an amount of about 15 wt % to about 30 wt %, based on a total weight of the treatment fluid. The treatment fluid may have a freeze point of about 0° F. to about −40° F. The contaminant may comprise a polymer-polyvalent metal ion complex. The polyvalent metal ion may comprise iron. The method may further comprise flowing the treatment fluid through a component of a fluid handling system to remove a contaminant from a surface of the component. The method may further comprise flowing the treatment fluid through a component of a fluid handling system to remove a contaminant from a surface of the component and washing or flushing the component with water or a flowback fluid to remove the contaminant from the component.

According to another aspect of the present disclosure, a treatment fluid for use in oil- and gas-field operations is provided. The treatment fluid includes gluconic acid, hydrogen peroxide, and optionally citric acid. The treatment fluid is configured to prevent or remove polymer-polyvalent metal ion complex deposits in a substrate.

According to other aspects of the present disclosure, the treatment fluid may include one or more of the following features. The treatment fluid may comprise from about 1 ppm to about 40 wt % of the gluconic acid and hydrogen peroxide combined, based on a total weight of the treatment fluid. The treatment fluid may further comprise methanol in an amount of about 15 wt % to about 30 wt %, based on a total weight of the treatment fluid. The treatment fluid may have a freeze point of about 0° F. to about −40° F. The treatment fluid may further comprise citric acid, wherein the gluconic acid is present in a greater amount than the citric acid. The treatment fluid may be substantially free of a chelating agent other than the gluconic acid and citric acid. The polymer-polyvalent metal ion complex may comprise iron.

According to another aspect of the present disclosure, a method of formulating a treatment fluid is provided. The method includes combining gluconic acid and hydrogen peroxide to form a treatment fluid. The method also includes optionally adding citric acid to the treatment fluid. The treatment fluid is configured to selectively bind to iron ions over calcium ions.

According to other aspects of the present disclosure, the method may include one or more of the following features. The method may further comprise adding methanol to the treatment fluid in an amount of about 15 wt % to about 38 wt %, based on a total weight of the treatment fluid, to achieve a freeze point of about 0° F. to about −40° F. The gluconic acid may be present in a greater amount than the citric acid. The treatment fluid may comprise from about 1 ppm to about 40 wt % of the gluconic acid and hydrogen peroxide combined, based on a total weight of the treatment fluid.

### DETAILED DESCRIPTION

For purposes of promoting an understanding of the principles of the disclosure, reference will now be made to the embodiments illustrated in the drawings, and specific language will be used to describe the same. It will nonetheless be understood that no limitation of the scope of the disclosure is intended by the illustration and description of certain embodiments of the disclosure. In addition, any alterations and/or modifications of the illustrated and/or described embodiment(s) are contemplated as being within the scope of the present disclosure. Further, any other applications of the principles of the disclosure, as illustrated and/or described herein, as would normally occur to one skilled in the art to which the disclosure pertains, are contemplated as being within the scope of the present disclosure.

The following terms shall have the following meanings:

The term “comprising” and derivatives thereof are not intended to exclude the presence of any additional component, step, or procedure, whether or not the same is disclosed herein. To avoid any doubt, all compositions claimed herein through the use of the term “comprising” may include any additional additive or compound unless stated to the contrary. In contrast, the term “consisting essentially of,” if appearing herein, excludes from the scope of any succeeding recitation any other component, step, or procedure, except those that are not essential to operability, and the term “consisting of,” if used, excludes any component, step or procedure not specifically delineated or listed. The term “or,” unless stated otherwise, refers to the listed members individually as well as in any combination.

The articles “a” and “an” are used herein to refer to one or more than one (i.e., to at least one) of the grammatical objects of the article. By way of example, “a peracid” means one peracid or more than one peracid. The phrases “in one embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same aspect. If the specification states a component or feature “may,” “can,” “could,” or “might” be included or have a characteristic, that particular component or feature is not required to be included or have the characteristic.

The term “about,” as used herein, can allow for a degree of variability in a value or range, for example, it may be within 10%, within 5%, or within 1% of a stated value or of a stated limit of a range.

The terms “preferred” and “preferably” refer to embodiments that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the present disclosure.

The term “optional” or “optionally” means that the subsequently described event, circumstance, or material may or may not occur or be present, and that the description includes instances where said event, circumstance, or material occurs or is present and instances where it does not occur or is not present.

Values expressed in a range format should be interpreted in a flexible manner to include not only the numerical values explicitly recited as the limits of the range but also include all of the individual numerical values or sub-ranges encompassed within that range as if each numerical value and sub-range is explicitly recited. For example, a range such as from 1 to 6 should be considered to have specifically disclosed sub-ranges, such as, from 1 to 3, from 2 to 4, from 3 to 6, etc., as well as individual numbers within that range, for example, 1, 2, 3, 4, 5, and 6. This applies regardless of the breadth of the range.

The term “substantially free” refers to a composition in which a particular compound or moiety is present in an amount that has no material effect on the composition. In some embodiments, “substantially free” may refer to a composition in which the particular compound or moiety is present in the composition in an amount of less than 2 wt. %, or less than 1 wt. %, or less than 0.5 wt. %, or less than 0.1 wt. %, or less than 0.05 wt. %, or even less than 0.01 wt. %, based on the total weight of the composition, or that no amount of that particular compound or moiety is present in the respective composition.

As used herein, the term “organic acid” refers to an organic compound that exhibits acidic properties. As used herein, the term “organic compound” refers to a carbon-based molecule derived from biological or synthetic sources. As used herein, the term “acid” refers to a substance that can donate protons (H⁺) or accept electron pairs. Non-limiting examples of organic acids include gluconic acid, citric acid, peroxy gluconic acid, and percitric acid.

As used herein, the term “polyhydroxy acid” refers to an organic acid with more than one hydroxyl group (—OH) attached to its carbon backbone in addition to a carboxyl group (—COOH). Non-limiting examples of polyhydroxy acid include gluconic acid.

As used herein, the term “peracid” may also be referred to as a “percarboxylic acid” or “peroxyacid.” Peracids also include peracids having at least one acidic group or peracidic group in the deprotonated form, for example, MH₂(peracid), M₂H(peracid), or M₃(peracid) where M may be sodium, potassium, ammonium, imidazolium, magnesium, or any other monocationic or polycationic species.

As used herein, the term “chelating agent” or “chelant” refers to a chemical compound that forms stable, multiple bonds with a metal ion, creating a chelate complex. Chelating agents can have multiple donor atoms (typically oxygen, nitrogen, or sulfur) with lone pairs of electrons, allowing them to bind to metal ions through coordinate bonds. Chelation may prevent the metal ion from reacting or precipitating.

The term “water” refers to a variety of water sources, such as freshwater, pond water, seawater, salt water or brine source, brackish water, or the like.

The term “process fluid” refers to any fluid utilized in, generated during, or resulting from an industrial, operational, or treatment process. Process fluids may serve as working media, carriers, reactants, or byproducts within a system and can include, but are not limited to: Flowback Fluids: Fluids that return to the surface after an industrial operation, such as hydraulic fracturing, containing water, chemical additives, and naturally occurring materials from the system or environment; Industrial Fluids: Liquids used in manufacturing, refining, or other industrial applications, including coolants, lubricants, and cutting fluids; Wastewater: Effluents from processes like water treatment, paper and pulp production, or disinfection activities, potentially containing dissolved solids, organic materials, or chemical residues; Recirculated Fluids: Reused fluids within closed-loop systems, such as those in water filtration, heat exchangers, or chemical processing; and Reaction Media: Fluids that facilitate chemical reactions, such as solvents or mixtures containing reactants and catalysts. Process fluids may vary in composition and properties depending on the specific application, and they can include both fresh and contaminated fluids, including mixtures of water, dissolved minerals, organic or inorganic materials, and chemical additives.

The term “flowback fluid(s)” is used herein to refer collectively and generically to returning fluids from industrial or operational processes, including but not limited to produced water, flowback water, returning treatment fluids, and the like. As used herein, “produced water” may refer to water produced from a wellbore that is not primarily a treatment fluid and may contain formation waters. Additionally, flowback fluids may include returning or recycled water from water treatment processes, paper and pulp processing operations, or disinfection activities, which may contain a range of contaminants, such as dissolved solids, organic materials, microbes, or chemical additives from the respective operations. In many cases, the composition of a flowback fluid is inexact or unknown.

The term “produced water,” as used herein, refers to water generated or returned from various industrial, operational, or natural processes. This includes, but is not limited to: Oil and Gas Operations: Water extracted from a wellbore, including formation water and flowback water, that is not primarily a treatment fluid; Water Treatment Processes: Water discharged or recycled from water treatment systems, which may include dissolved minerals, residual treatment chemicals, or organic and microbial contaminants; Paper and Pulp Processing: Water used or recovered during paper manufacturing or pulp processing, often containing cellulose fibers, organic residues, lignin residues, inks, dyes, bleaching agents, and other processing chemicals; Disinfection Activities: Water generated from cleaning, sanitizing, or sterilizing activities in residential, commercial, industrial, or medicinal contexts, which may contain microbial residues, cleaning agents, or byproducts of disinfection.

The term “wt. %” means weight percent.

The term “gpt” means gallons per thousand gallons, and the term “ppm” means parts per million.

The term “substrate” refers to any material, surface, or structural component capable of interacting with a treatment fluid. Substrates may include both external surfaces and internal structures, such as porous materials, geological formations, filter membranes, fibers, and the like.

The substrate may be rigid or flexible, porous or non-porous, and may include but is not limited to: Industrial equipment surfaces, such as metal, plastic, or ceramic components of machinery or pipelines; Environmental surfaces, such as geological formations, wellbores, or storage tanks; Processing materials, such as fibers, films, or sheets in paper and pulp processing; Surfaces involved in water treatment, including filtration membranes or system components; Surfaces used in disinfection, including walls, floors, or tools in industrial, medical, or residential environments.

A rigid surface refers to a solid, immovable plane with a specific level of structural support. For example, a rigid surface may be a substantially planar, non-deformable surface configured to provide structural support for an object. An object on a rigid surface may remain stationary and is supported entirely above the surface without deformation. A rigid surface may be configured for mechanical stability. A rigid surface may be characterized by fixed dimensions and resistance to external forces. A flexible surface is capable of deformation under the weight of an object. A flexible surface may have a variable contour due to external disturbances.

As used herein, the term ‘rigid substrate’ refers to a material or surface that retains its shape and does not deform significantly under applied force during typical use conditions. Rigid substrates are characterized by high stiffness, typically quantified by a high modulus of elasticity, and are commonly used in applications requiring structural support or stability. Examples include, but are not limited to, metals, ceramics, glass, rigid plastics, and composite materials.” A rigid substrate may have high stiffness, such as resistance to bending or deformation under force or high Young's modulus (e.g., metals, ceramics). A rigid substrate may be used to provide structural support, load-bearing components, or surfaces requiring dimensional stability (e.g., pipes, equipment surfaces, or machine components).

As used herein, the term ‘flexible substrate’ refers to a material or surface capable of bending, flexing, or deforming significantly without cracking or breaking under applied force during typical use conditions. Flexible substrates are characterized by low stiffness, typically quantified by a lower modulus of elasticity, and are commonly used in applications requiring flexibility, conformability, or adaptability. Examples include, but are not limited to, thin films, elastomers, textiles, and flexible plastics.” A flexible substrate may have low stiffness, such as significant deformation or bending under force or lower Young's modulus (e.g., elastomers, textiles). A flexible substrate may be used to provide conformable surfaces, wrapping or coating applications, and dynamic environments (e.g., membranes, flexible packaging).

The present disclosure provides a method and treatment fluid for use in various applications, such as oil- and gas-field operations (e.g., wellbore and subterranean formations), water treatment, paper and pulp processing, and disinfection. The treatment fluid comprises an oxidation product or oxidation derivative of a carbohydrate, such as the reaction product of hydrogen peroxide with gluconic acid and, optionally, citric acid. This unique combination of components offers potential advantages over conventional treatment fluids, particularly in the prevention or removal of polymer-polyvalent metal ion complex deposits.

Polymer-polyvalent metal ion complexes, particularly those involving iron, can cause significant operational issues in a variety of industrial and environmental applications, including oil- and gas-field operations, water treatment, paper and pulp processing, and disinfection processes. In oil- and gas-field operations, these complexes can form deposits or scales in the wellbore, production equipment, and within the formation itself, leading to reduced production rates, equipment damage, and decreased operational efficiency. Similarly, in water treatment systems, these deposits can impair filtration membranes, clog pipelines, and reduce the efficacy of water purification processes. In paper and pulp processing, such deposits can accumulate on machinery, affecting production quality and increasing maintenance downtime. In disinfection processes, polymer-metal ion complexes can contribute to fouling of surfaces, reduce the effectiveness of antimicrobial treatments, and create biofilm-supporting structures. These challenges highlight the importance of effective treatments to prevent or remove these deposits across diverse industries.

The treatment fluid disclosed herein is configured to selectively bind to iron ions over other ions such as calcium, thereby offering a more targeted approach to preventing or removing these problematic deposits. Furthermore, the treatment fluid may exhibit improved solubility characteristics, particularly in cold climate formulations containing methanol, due to the presence of alcohol groups in gluconic acid. The inclusion of methanol in the treatment fluid may be advantageous in certain operational contexts, particularly in cold climates. For instance, the methanol may help to lower the freeze point of the treatment fluid, thereby enhancing its usability and effectiveness in cold weather conditions. In some cases, the treatment fluid may have a freeze point of about 0° F. to about −40° F. (about −18° C. to about −40° C.), depending on the specific concentration of methanol used.

In addition to its use in wellbore and subterranean formations, the treatment fluid may also be used in other aspects of oil- and gas-field operations, such as flowing through components of a fluid handling system to remove contaminants from component surfaces. Furthermore, the treatment fluid is applicable to other industrial and environmental processes, including water treatment, paper and pulp processing, and disinfection. In water treatment, the fluid may flow through system components, such as pipelines, filtration units, or storage tanks, to remove scale, biofilms, or other deposits from surfaces. In paper and pulp processing, the fluid can be applied to machinery or processing equipment to clean surfaces fouled by polymer-metal ion complexes, biofilms, or scale, thereby maintaining operational efficiency. In disinfection, the fluid can treat surfaces or systems to remove biofilms, reduce microbial contamination, and prevent residue accumulation that can harbor microorganisms.

The present disclosure further provides a method of formulating the treatment fluid, which involves combining an oxidation product or oxidation derivative of a carbohydrate, such gluconic acid, and hydrogen peroxide, and optionally adding citric acid. This method allows for the production of a treatment fluid that is tailored to the specific needs of a given operation, offering flexibility and adaptability in a variety of operational contexts.

In summary, the present disclosure provides a novel treatment fluid and associated methods that offer potential advantages in the prevention and removal of polymer-polyvalent metal ion complex deposits across a range of industrial and environmental applications, including oil- and gas-field operations, water treatment, paper and pulp processing, and disinfection. In oil- and gas-field operations, the treatment fluid can mitigate deposits in wellbores, subterranean formations, and fluid handling systems, improving efficiency and equipment longevity. In water treatment, it addresses fouling in pipelines, filtration systems, and storage tanks by preventing or removing complex deposits and biofilms. In paper and pulp processing, the fluid can clean processing equipment and machinery fouled by polymer-metal ion complexes or other deposits, reducing downtime and enhancing production quality. In disinfection, it can remove biofilms, microbial contamination, and residues from surfaces or systems, ensuring cleaner environments and more effective disinfection.

In some aspects, the treatment fluid may include gluconic acid and hydrogen peroxide. Gluconic acid is an organic compound derived from glucose through the oxidation of the aldehyde group at carbon-1 into a carboxyl group. Gluconic acid is derived from glucose, so it retains the hydroxyl (—OH) groups found in the glucose molecule. Specifically, gluconic acid has five hydroxyl groups attached to the carbon atoms, making it an alcohol-containing compound. However, the aldehyde group of glucose (at carbon-1) is oxidized to a carboxylic acid (—COOH), forming gluconic acid. In some examples, the treatment fluid also may include glucaric acid or other oxidation product or oxidation derivative of glucose or gluconic acid.

In other examples, the treatment fluid may include hydrogen peroxide and the oxidation product or oxidation derivative of other carbohydrates, including, but not limited to, starch, polysaccharides, saccharides, sugars, sugar alcohols, sugar acids, glucose, or the like. For example, the treatment fluid may be derived from one or more hydrolysed carbohydrates that are further oxidized to define at least one carboxyl group or an aldaric acid. In this way, various renewable feedstocks including at least one carbohydrate may be used to form the treatment fluids described herein. For this reason, as used herein, gluconic acid may refer to other select oxidation products or oxidation derivatives of other carbohydrates.

However, gluconic acid may provide other unique properties for select applications relative to other oxidation products or oxidation derivatives of other carbohydrates. For example, gluconic acid, having only one acid site as a single carboxyl group, may be more compatible with other fluid constituents, such as friction reducers, by having less affect on pH relative to other treatment fluid constituents having a greater number of acid sites, such as constituents having two or three acid sites. In practice, a treatment fluid including gluconic acid may be more compatible in water sources having wider range of natural buffering capacity (e.g., pond water with a greater bicarbonate loading compared to spring water) relative to treatment fluids having constituents with two or more acid sites. For this reason, using gluconic acid in the treatment fluid presents greater effectiveness over a wider variety of water source conditions relative to a treatment fluid including only citric acid.

The gluconic acid, oxidation product or oxidation derivative of other carbohydrates, may serve as a chelating agent, selectively binding to iron ions over other ions such as calcium. This selective binding may be particularly advantageous in preventing or removing polymer-polyvalent metal ion complex deposits, which often involve iron ions. The hydrogen peroxide may serve as an oxidizing agent, further enhancing the treatment fluid's ability to break down and remove such deposits.

In some cases, the treatment fluid may further include citric acid. The citric acid may also serve as a chelating agent, potentially enhancing the treatment fluid's ability to bind to and remove iron ions. The inclusion of citric acid may provide additional flexibility in tailoring the treatment fluid to the specific needs of a given operation. For example, citric acid is a triprotic acid with three carboxyl groups and one hydroxyl group, providing additional sites for binding with metal ions.

In some embodiments, the treatment fluid may be substantially free of any chelating agent other than the gluconic acid and/or citric acid. This may help to ensure that the treatment fluid's chelating activity is primarily directed towards iron ions, thereby maximizing its effectiveness in preventing or removing polymer-polyvalent metal ion complex deposits.

According to one embodiment, the present disclosure is directed to a method for forming a composition containing peroxy gluconic acid and to a composition containing peroxy gluconic acid formed by such a method, the composition being suitable for use in various treatment fluids for oil- and gas-field operations, water treatment, paper and pulp processing, general disinfection, etc. The method may generally include contacting gluconic acid with hydrogen peroxide in the presence of water or a flowback fluid to form a composition that includes an oxidation product or oxidative derivative of gluconic acid, such as glucaric acid, a peroxy gluconic acid, and/or a peroxy glucaric acid. The peroxy gluconic acid may be a monoperoxy gluconic acid (i.e., single peroxide group added to the gluconic acid molecule) or a polyperoxy gluconic acid (i.e., multiple peroxide groups added to the gluconic acid molecule). The peroxy gluconic acid may be a 2-peroxy gluconic acid, a 3-peroxy gluconic acid, a 5-peroxy gluconic acid, or any mixture thereof. In one embodiment, the peroxy gluconic acid is a 2-peroxy gluconic acid. (It is generally understood that when there is an excess of gluconic acid, the composition that is formed will also comprise gluconic acid). In 2-peroxy gluconic acid, the hydroxyl group (—OH) at the C2 position in gluconic acid is replaced by a peroxide group (—OOH). Similarly, in 3-peroxy gluconic acid, the hydroxyl group at the third carbon (C3) is replaced with a peroxide group (—OOH). In 5-peroxy gluconic acid, the hydroxyl group at the fifth carbon (C5) is replaced with a peroxide group (—OOH). The product of contacting gluconic acid with hydrogen peroxide in the presence of water or a flowback fluid may be a composition that includes 2-peroxy gluconic acid, 3-peroxy gluconic acid, and 5-peroxy gluconic acid, which may be the result of a reaction dominated by C2 substitution. In such a composition, the total product may include from about 60 to about 80% 2-peroxyl gluconic acid, about 10 to about 20% 3-peroxy gluconic acid, and about 10 to about 15% 5-peroxy gluconic acid. These percentages are approximate and may vary based on reaction conditions, such as pH, temperature, and reactant concentrations. The composition may include water, oxygen, gluconic acid residues, organic byproducts (e.g., oxidized organics, residual radicals, carboxylic acids, aldehydes, ketones, etc.), metal peroxides if metals are present (e.g., metal ions (e.g., Fe²⁺, Cu²⁺) from flowback fluid can react with hydrogen peroxide to form peroxides or chelated complexes), decomposition products of peroxy gluconic acid if under harsh conditions of extreme pH or elevated temperatures, etc. Metal ions (e.g., Fe²⁺, Cu²⁺) in flowback fluid can catalyze the decomposition of hydrogen peroxide, leading to the formation of oxygen gas and radicals. These radicals may react with gluconic acid or other components in the mixture, potentially generating chelated complexes or other byproducts. The presence and composition of organic byproducts depend heavily on the impurities present in the flowback fluid, such as dissolved organics or hydrocarbons. The composition may depend on whether contacting gluconic acid with hydrogen peroxide is in the presence of water or a flowback fluid; whether there is excess reactants, such as hydrogen peroxide, pH conditions, and temperature; etc.

According to one embodiment, the present disclosure is directed to a method for forming a composition containing peroxy gluconic and percitric acid and to a composition containing peroxy gluconic acid and percitric acid formed by such a method, the composition being suitable for use in various treatment fluids for oil- and gas-field operations, water treatment, paper and pulp processing, general disinfection, etc. The method may generally include contacting gluconic acid and citric acid with hydrogen peroxide in the presence of water or a flowback fluid to form a composition that includes peroxy gluconic acid and percitric acid. The peroxy gluconic acid may be a 2-peroxy gluconic acid, a 3-peroxy gluconic acid, a 5-peroxy gluconic acid, or any mixture thereof. In one embodiment, the peroxy gluconic acid is a 2-peroxy gluconic acid. (It is generally understood that when there is an excess of gluconic acid, the composition that is formed will also comprise gluconic acid). The percitric acid may be monopercitric acid, dipercitric acid, tripercitric acid or any mixture thereof. In one embodiment, the percitric acid is a monopercitric acid; however, by adjusting the mole ratios of hydrogen peroxide to citric acid, dipercitric acid or tripercitric acid may be formed. (It is generally understood that when there is an excess of citric acid, the composition that is formed will also comprise citric acid). U.S. patent application Ser. No. 18/578,654, entitled “Use of Percitirc Acid in Oil- and Gas-Field Operations,” describes using percitric acid for oil- and gas-operations, the entirety of which is incorporated by reference herein.

In some embodiments, before the contacting step, the gluconic acid can be provided in a composition that comprises gluconic acid, for example, an aqueous solution that comprises gluconic acid. In other embodiments, before the contacting step, the gluconic acid can be provided in a composition that comprises a substance that generates gluconic acid upon contact with water. Any suitable substance that generates gluconic acid can be used in the present methods. The substance can be a salt of gluconate, for example, a sodium or calcium salt of gluconate, or an ester of gluconate. Gluconic acid can be made through biological fermentation, chemical oxidation, electrochemical oxidation, enzymatic conversion, and oxidation with hydrogen peroxide of glucose.

In some embodiments, before the contacting step, the hydrogen peroxide can be provided in a composition that comprises hydrogen peroxide, for example, an aqueous solution that comprises hydrogen peroxide. In other embodiments, before the contacting step, the hydrogen peroxide can be provided in a composition that comprises a substance that generates hydrogen peroxide upon contact with water. Any suitable substance that generates hydrogen peroxide can be used in the present methods and in some embodiments can comprise a precursor of hydrogen peroxide. For example, the precursor of hydrogen peroxide can be sodium percarbonate, sodium perborate, sodium persulfate, urea hydrogen peroxide, or PVP-hydrogen peroxide.

In still another embodiment, an oxygen-containing gas can be supplied to an electrolysis unit to produce peroxide species (e.g. hydrogen peroxide, peroxide ions, peroxide radicals) which can be contacted with gluconic acid and/or citric acid.

In some aspects, the gluconic acid, hydrogen peroxide, and optionally citric acid may be combined in any suitable proportions to form the treatment fluid. The specific proportions may be selected based on various factors, such as the specific operational context, the nature of the deposits to be prevented or removed, and the desired properties of the treatment fluid. For example, in some cases, the gluconic acid and hydrogen peroxide may be combined in a ratio that optimizes the treatment fluid's chelating and oxidizing activities. In other cases, the gluconic acid, hydrogen peroxide, and citric acid may be combined in a ratio that provides a desired balance of chelating activity, oxidizing activity, and overall treatment fluid stability.

In some aspects, the treatment fluid may include about 1 ppm to about 95 wt. % of total acid (e.g., gluconic acid and/or citric acid), such as about 1 ppm, 30 ppm, 250 ppm, 15 wt. %, 30 wt. %, or 40 wt. %.

In some aspects, the treatment fluid may comprise from about 1 ppm to about 40 wt % of the gluconic acid and hydrogen peroxide combined, based on a total weight of the treatment fluid. This range may provide a suitable balance between the chelating and oxidizing activities of the treatment fluid, while also ensuring sufficient solubility and stability of the treatment fluid in various operational contexts. For example, at lower concentrations, such as in the ppm range, the treatment fluid may still effectively prevent or remove polymer-polyvalent metal ion complex deposits, while also being compatible with other components of the fluid handling system or wellbore environment. On the other hand, at higher concentrations, such as in the wt % range, the treatment fluid may provide enhanced chelating and oxidizing activities, which may be particularly advantageous in operations involving heavy deposits or challenging wellbore conditions. In some cases, the specific concentration of the gluconic acid and hydrogen peroxide in the treatment fluid may be selected based on factors such as the nature and extent of the deposits to be treated, the specific operational context, and the desired properties of the treatment fluid.

In some aspects, the treatment fluid may include citric acid in addition to gluconic acid. The relative concentration of gluconic acid to citric acid may vary depending on the specific needs of the operation. For instance, in some cases, the gluconic acid may be present in a greater amount than the citric acid in the treatment fluid. This may be advantageous in situations where a higher selectivity for iron ions is desired, as gluconic acid has been found to bind more selectively to iron ions compared to citric acid.

In some cases, the treatment fluid may comprise blends of gluconic acid and citric acid in various ratios. Before the contacting step, the molar ratio of citric acid to gluconic acid may range between about 0:100 to about 99:1, such as about 5:95 to about 95:5, about 10:90 to about 90:10, about 15:85 to about 85:15, about 20:80 to about 80:20, about 25:75 to about 75:25, about 30:70 to about 70:30, about 40:60 to about 60:40, or other suitable molar ratio. In other embodiments, the molar ratio of citric acid to gluconic acid may be about 20:80, about 40:60, about 60:40, about 80:20, or other suitable molar ratio. The specific molar ratio may be selected based on factors such as the nature and extent of the deposits to be treated, the specific operational context, and the desired properties of the treatment fluid. For instance, a higher molar ratio of gluconic acid to citric acid may be used in operations involving heavy iron deposits, while a lower molar ratio may be used in operations involving lighter deposits or less challenging wellbore conditions.

In some cases, the treatment fluid may comprise blends of gluconic acid and citric acid in various ratios. Before the contacting step, the weight percentage ratio of citric acid to gluconic acid may range between about 0:100 to about 99:1, such as about 5:95 to about 95:5, about 10:90 to about 90:10, about 15:85 to about 85:15, about 20:80 to about 80:20, about 25:75 to about 75:25, about 30:70 to about 70:30, about 40:60 to about 60:40, or other suitable weight percentage ratio. In other embodiments, the weight percentage ratio of citric acid to gluconic acid may be about 20:80, about 40:60, about 60:40, about 80:20, or other suitable weight percentage ratio. The specific weight percentage ratio may be selected based on factors such as the nature and extent of the deposits to be treated, the specific operational context, and the desired properties of the treatment fluid. For instance, a higher weight percentage ratio of gluconic acid to citric acid may be used in operations involving heavy iron deposits, while a lower weight percentage ratio may be used in operations involving lighter deposits or less challenging wellbore conditions.

In some embodiments, the treatment fluid may comprise a mixture of gluconic acid, citric acid, and hydrogen peroxide, with the gluconic acid present in a greater amount than the citric acid. This combination may provide a balance of chelating activity, oxidizing activity, and overall treatment fluid stability, while also offering a high selectivity for iron ions.

In one embodiment, the molar ratio of hydrogen peroxide to citric acid is chosen such that the composition includes at least one of monopercitric acid, dipercitric acid or tripercitric acid, preferably monopercitric acid.

In one embodiment, the molar ratio of hydrogen peroxide to gluconic acid is chosen such that the composition includes at least one peroxy gluconic acid, such as 2-peroxy gluconic acid, 3-peroxy gluconic acid, or 5-peroxy gluconic acid.

Before the contacting step, the molar ratio of hydrogen peroxide to citric acid may range between about 0.01:1 to about 100:1, such as about 0.02:1 to about 50:1 or about 0.05:1 to about 20:1 or about 0.07:1 to about 10:1 or about 0.1:1 to about 3:1. In other embodiments, the molar ratio of hydrogen peroxide to citric acid may range between about 0.5:1 to about 2:1 or between about 0.75:1 to about 1.5.:1 or between about 0.95:1 to about 1.05:1 or about 1:1. In one embodiment the molar ratio of hydrogen peroxide to citric acid is chosen such that the composition includes at least one of monopercitric acid, dipercitric acid or tripercitric acid, preferably monopercitric acid.

In some embodiments, a base (for example, NaOH or KOH) may be added to sufficiently raise the pH so that the composition includes a conjugate base of percitric acid.

The present method above can be conducted at any suitable temperature. In some embodiments, the present method can be conducted at a temperature ranging from about −2° C. to about 70° C. In other embodiments, the present method can be conducted under ambient conditions. In still other embodiments, the present method can be conducted under heating, for example, at a temperature ranging from about 30° C. to about 70° C.

The composition can comprise any suitable concentration of peroxy gluconic acid. In some embodiments, the composition contains from about 0.001 wt. % to about 70 wt. % peroxy gluconic acid, based on the total weight of the composition, for example, greater than about 0.001 wt. %, greater than about 0.1 wt. %, greater than about 0.5 wt. %, greater than about 1 wt. %, greater than about 5 wt. %, greater than about 10 wt. %, greater than about 15 wt. %, greater than about 20 wt. %, greater than about 25 wt. %, greater than about 30 wt. %, greater than about 35 wt. %, greater than about 40 wt. % or greater than about 45 wt. % peroxy gluconic acid, or about 50 wt. % or greater, based on the total weight of the composition. In still other embodiments, the composition may contain from about 0.5 wt. % to about 50 wt. %, or from about 1 wt. % to about 40 wt. %, or from about 10 wt. % to about 30 wt. % or from about 15 wt. % to about 25 wt. % or from about 18 wt. % to about 22 wt. % of peroxy gluconic acid, based on the total weight of the composition.

The composition can comprise any suitable concentration of percitric acid. In some embodiments, the composition contains from about 0.001 wt. % to about 70 wt. % percitric acid, based on the total weight of the composition, for example, greater than about 0.001 wt. %, greater than about 0.1 wt. %, greater than about 0.5 wt. %, greater than about 1 wt. %, greater than about 5 wt. %, greater than about 10 wt. %, greater than about 15 wt. %, greater than about 20 wt. %, greater than about 25 wt. %, greater than about 30 wt. %, greater than about 35 wt. %, greater than about 40 wt. % or greater than about 45 wt. % percitric acid, or about 50 wt. % or greater, based on the total weight of the composition. In still other embodiments, the composition may contain from about 0.5 wt. % to about 50 wt. %, or from about 1 wt. % to about 40 wt. %, or from about 10 wt. % to about 30 wt. % or from about 15 wt. % to about 25 wt. % or from about 18 wt. % to about 22 wt. % of percitric acid, based on the total weight of the composition.

The composition can also include any suitable concentration of hydrogen peroxide. In some embodiments, the composition can contain about 35 wt. % or less hydrogen peroxide, based on the total weight of the composition, for example, less than about 30 wt. %, less than about 25 wt. %, less than about 20 wt, %, less than about 15 wt. %, less than about 10 wt. %, less than about 9 wt. %, less than about 8 wt. %, less than about 7 wt. %, less than about 6 wt. %, or less than about 5 wt. %, less than about 4.5 wt. %, less than about 4 wt, %, less than about 3.5 wt. %, less than about 3 wt. %, less than about 2.5 wt. %, less than about 2 wt. %, less than about 1.5 wt. %, less than about 1 wt. %, less than about 0.9 wt. %, less than about 0.8 wt. %, less than about 0.7 wt. %, less than about 0.6 wt. %, less than about 0.5 wt. %, less than about 0.4 wt. %, less than about 0.3 wt. %, less than about 0.2 wt. %, less than about 0.1 wt. %, less than about 0.05 wt. %, less than about 0.01 wt. %, less than about 0.005 wt. %, or less than about 0.001 wt. % of hydrogen peroxide, based on the total weight of the composition. In some embodiments, the composition is substantially free of hydrogen peroxide.

In some embodiments, the composition can also include any suitable concentration of gluconic acid. In some embodiments, the composition can contain about 70 wt. % or less gluconic acid, based on the total weight of the composition, for example, less than about 60 wt. %, less than about 50 wt. %, less than about 40 wt, %, less than about 30 wt. %, less than about 20 wt. %, less than about 10 wt. %, less than about 8 wt. %, less than about 7 wt. %, less than about 6 wt. %, or less than about 5 wt. %, less than about 4.5 wt. %, less than about 4 wt, %, less than about 3.5 wt. %, less than about 3 wt. %, less than about 2.5 wt. %, less than about 2 wt. %, less than about 1.5 wt. %, less than about 1 wt. %, less than about 0.9 wt. %, less than about 0.8 wt. %, less than about 0.7 wt. %, less than about 0.6 wt. %, less than about 0.5 wt. %, less than about 0.4 wt. %, less than about 0.3 wt. %, less than about 0.2 wt. %, less than about 0.1 wt. %, less than about 0.05 wt. %, less than about 0.01 wt. %, less than about 0.005 wt. %, or less than about 0.001 wt. % of gluconic acid, based on the total weight of the composition. In some embodiments, the composition is substantially free of gluconic acid.

In some embodiments, the composition can also include any suitable concentration of citric acid. In some embodiments, the composition can contain about 70 wt. % or less citric acid, based on the total weight of the composition, for example, less than about 60 wt. %, less than about 50 wt. %, less than about 40 wt, %, less than about 30 wt. %, less than about 20 wt. %, less than about 10 wt. %, less than about 8 wt. %, less than about 7 wt. %, less than about 6 wt. %, or less than about 5 wt. %, less than about 4.5 wt. %, less than about 4 wt, %, less than about 3.5 wt. %, less than about 3 wt. %, less than about 2.5 wt. %, less than about 2 wt. %, less than about 1.5 wt. %, less than about 1 wt. %, less than about 0.9 wt. %, less than about 0.8 wt. %, less than about 0.7 wt. %, less than about 0.6 wt. %, less than about 0.5 wt. %, less than about 0.4 wt. %, less than about 0.3 wt. %, less than about 0.2 wt. %, less than about 0.1 wt. %, less than about 0.05 wt. %, less than about 0.01 wt. %, less than about 0.005 wt. %, or less than about 0.001 wt. % of citric acid, based on the total weight of the composition. In some embodiments, the composition is substantially free of citric acid.

The present disclosure provides for the use of gluconic acid, either alone or in combination with citric acid, in a treatment fluid for oil- and gas-field operations, water treatment, paper and pulp processing, general disinfection, etc. This offers potential advantages in the prevention and removal of polymer-polyvalent metal ion complex deposits, particularly those involving iron ions. The specific formulation of the treatment fluid, including the relative concentrations of gluconic acid and citric acid, may be tailored to the specific needs of a given operation, offering flexibility and adaptability in a variety of operational contexts.

In some aspects, the treatment fluid may be configured to selectively bind to iron ions over calcium ions when used in a wellbore or a subterranean formation. This selective binding may be attributed to the presence of gluconic acid in the treatment fluid. Gluconic acid, as a chelating agent, may exhibit a higher selectivity for iron ions compared to other ions such as calcium. This selective binding to iron ions may be particularly advantageous in preventing or removing polymer-polyvalent metal ion complex deposits, which often involve iron ions.

In some cases, the selective binding of gluconic acid to iron ions over calcium ions may enhance the effectiveness of the treatment fluid in preventing or removing polymer-polyvalent metal ion complex deposits. For instance, by selectively binding to iron ions, the gluconic acid may help to prevent the formation of these deposits in the first place, or aid in their removal once formed. This may be particularly beneficial in operations involving heavy iron deposits or challenging conditions in the substrate, for example, wellbore.

In other aspects, the selective binding properties of gluconic acid may also contribute to the overall stability of the treatment fluid. For example, by selectively binding to iron ions, the gluconic acid may help to prevent unwanted interactions with other ions such as calcium, which could potentially interfere with the performance of the treatment fluid. This may result in a treatment fluid that is more stable and reliable in a variety of operational contexts.

In summary, the use of gluconic acid in the treatment fluid may provide a more targeted approach to preventing or removing polymer-polyvalent metal ion complex deposits in oil- and gas-field operations, water treatment, paper and pulp processing, disinfection, etc. This may offer potential advantages over conventional treatment fluids, particularly in terms of selectivity for iron ions and overall treatment fluid stability.

In some aspects, the treatment fluid may be used to treat a substrate, such as a wellbore or subterranean formation. This may involve contacting the substrate, such as at least one of the wellbore and the subterranean formation, with the treatment fluid for an effective amount of time. The specific duration of contact may be selected based on various factors, such as the nature and extent of the deposits to be treated, the specific operational context, and the desired properties of the treatment fluid. For example, in some cases, the treatment fluid may be contacted with the substrate, such as the wellbore or subterranean formation, for a period of time sufficient to remove a contaminant, reduce a microbial population, or prevent the formation of a polymer-polyvalent metal ion complex deposit.

In some cases, the treatment fluid may be used for remediation purposes. For instance, the treatment fluid may be used to remove existing polymer-polyvalent metal ion complex deposits in a substrate, such as a wellbore or subterranean formation. This may involve flowing the treatment fluid through the substrate, such as the wellbore or the subterranean formation, allowing the treatment fluid to contact the deposits, and then removing the treatment fluid along with the dissolved or dislodged deposits. The specific procedure for using the treatment fluid in this manner may be tailored to the specific needs of the operation, taking into account factors such as the nature and extent of the deposits, the configuration of the substrate, such as the wellbore or subterranean formation, and the desired properties of the treatment fluid.

In other aspects, the treatment fluid may be used in a preventative manner to inhibit the formation of polymer-polyvalent metal ion complex deposits in the first place. This may involve introducing the treatment fluid into the wellbore or subterranean formation prior to or during an operation that could potentially lead to the formation of such deposits. The treatment fluid may then bind to iron ions present in the substrate, such as the wellbore or subterranean formation, thereby preventing these ions from participating in the formation of polymer-polyvalent metal ion complex deposits. This preventative use of the treatment fluid may be particularly advantageous in operations involving heavy iron deposits or challenging conditions in the substrate, such as the wellbore.

In some aspects, the treatment fluid may be used to contact a substrate, such as at least one of the wellbore and the subterranean formation, for an effective amount of time. This contact time may be sufficient to achieve at least one of the following: remove a contaminant, reduce a microbial population, or prevent the formation of a polymer-polyvalent metal ion complex deposit. The specific duration of contact may be selected based on various factors, such as the nature and extent of the deposits to be treated, the specific operational context, and the desired properties of the treatment fluid.

In some cases, the contaminant that the treatment fluid is designed to remove may comprise a polymer-polyvalent metal ion complex. These complexes can form when polymers used in oil and gas operations, water treatment, paper and pulp processing, general disinfection, etc. interact with polyvalent metal ions, such as iron, present in the substrate (e.g., wellbore or subterranean formation). These complexes can cause significant operational issues, including the formation of deposits or scales that can reduce production rates, damage equipment, and decrease overall operational efficiency.

In some embodiments, the polyvalent metal ion involved in the formation of the polymer-polyvalent metal ion complex may comprise iron. Iron ions are commonly present in substrates, such as wellbore and subterranean formations, due to the natural mineral composition of these environments. When these iron ions interact with polymers, they can form polymer-iron ion complexes that can precipitate out of the fluid and form deposits. The treatment fluid disclosed herein, comprising gluconic acid and hydrogen peroxide, and optionally citric acid, may be particularly effective in preventing or removing these iron-containing deposits due to its ability to selectively bind to iron ions over other ions such as calcium.

In some aspects, the treatment fluid may be flowed through a component of a fluid handling system to remove a contaminant from a surface of the component. The fluid handling system may include any system or equipment used in oil- and gas-field operations, water treatment processes, paper and pulp processing, or disinfection activities. Examples include, but are not limited to: Oil- and gas-field operations: Drilling rigs, production equipment, pipelines, storage tanks, and the like; Water Treatment Processes: Filters, clarifiers, reverse osmosis systems, pipelines, tanks, and distribution networks; Paper and Pulp Processing: Piping systems, paper machines, dewatering equipment, storage tanks, and pumps used in the transport and processing of pulp or water; and Disinfection Activities: Sterilization systems, cleaning equipment, spray systems, piping, valves, and tanks used in residential, commercial, industrial, or medicinal disinfection operations. The component may be any part of the fluid handling system that comes into contact with fluids used in the operations, such as pipes, valves, pumps, spray nozzles, filters, or other surfaces that may accumulate contaminants such as mineral scales, organic residues, biofilms, or chemical deposits.

The contaminant may be any undesirable substance that can accumulate on the surface of the component, such as scale, rust, microbial growth, or polymer-polyvalent metal ion complex deposits. In some cases, the contaminant may comprise a polymer-polyvalent metal ion complex, particularly those involving iron ions. These complexes can form when polymers used in oil and gas operations, water treatment, paper and pulp processing, or disinfection, interact with polyvalent metal ions, such as iron, present in the substrate, such as wellbore or subterranean formation in oil and gas operations. These complexes can cause significant operational issues, including the formation of deposits or scales that can reduce production rates, damage equipment, and decrease overall operational efficiency.

In some cases, the treatment fluid may be flowed through the component for an effective amount of time to remove the contaminant. The specific duration of flow may be selected based on various factors, such as the nature and extent of the contaminant, the configuration of the component, and the desired properties of the treatment fluid. For example, a longer flow time may be used for components with heavy deposits or complex configurations, while a shorter flow time may be used for components with lighter deposits or simpler configurations.

In some embodiments, the treatment fluid may be flowed through the component at a specific flow rate to effectively remove the contaminant. The specific flow rate may be selected based on various factors, such as the nature and extent of the contaminant, the configuration of the component, and the desired properties of the treatment fluid. For example, a higher flow rate may be used for components with heavy deposits or complex configurations, while a lower flow rate may be used for components with lighter deposits or simpler configurations.

In some aspects, the treatment fluid may be recirculated through the component multiple times to enhance the removal of the contaminant. This recirculation may help to ensure that all areas of the component are thoroughly contacted by the treatment fluid, thereby maximizing its effectiveness in removing the contaminant.

In some cases, the treatment fluid may be followed by a rinse or flush with water or another suitable fluid to remove any residual treatment fluid and dislodged contaminant from the component. This rinse or flush may help to ensure that the component is cleaned and ready for further use in oil- and gas-field operations.

In some aspects, after the treatment fluid has been flowed through the component of the fluid handling system, the component may be washed or flushed with water or a flowback fluid to remove the contaminant from the component. This washing or flushing step may help to ensure that any residual treatment fluid and dislodged contaminant are effectively removed from the component, thereby enhancing the overall cleanliness and operational efficiency of the component. Effectively removed may include dislodging a selected percentage of contaminants to achieve a selected operational efficiency of a component.

In some cases, the washing or flushing step may be performed immediately after the treatment fluid has been flowed through the component. In other cases, there may be a delay between the flowing of the treatment fluid and the washing or flushing step, depending on the specific operational context and the nature of the contaminant.

The water or flowback fluid used for the washing or flushing step may be selected based on various factors, such as the specific operational context, the nature of the contaminant, and the desired properties of the washing or flushing fluid. For example, in some cases, water may be used for its simplicity and availability. In other cases, a flowback fluid may be used, particularly if the flowback fluid has properties that are beneficial for the removal of the contaminant.

In some embodiments, the washing or flushing step may be performed at a specific flow rate to effectively remove the contaminant. The specific flow rate may be selected based on various factors, such as the nature and extent of the contaminant, the configuration of the component, and the desired properties of the washing or flushing fluid. For example, a higher flow rate may be used for components with heavy deposits or complex configurations, while a lower flow rate may be used for components with lighter deposits or simpler configurations.

In some aspects, the washing or flushing step may be repeated multiple times to enhance the removal of the contaminant. This repetition may help to ensure that all areas of the component are cleaned, thereby maximizing the effectiveness of the washing or flushing step.

In summary, the present disclosure provides for the use of a treatment fluid comprising gluconic acid and hydrogen peroxide, and optionally citric acid, in combination with a subsequent washing or flushing step, to effectively remove contaminants from components of a fluid handling system in oil- and gas-field operations, water treatment, paper and pulp processing, disinfection, etc. This combination of steps offers potential advantages in terms of operational efficiency, flexibility, and adaptability in a variety of operational contexts.

In some aspects, the treatment fluid may be substantially free of chelating agents other than gluconic acid and citric acid. This configuration may help to ensure that the treatment fluid's chelating activity is primarily directed towards iron ions, thereby maximizing its effectiveness in preventing or removing polymer-polyvalent metal ion complex deposits.

In some cases, particularly in cold climate formulations containing methanol, the treatment fluid may be formulated to prevent esterification reactions between the acid and methanol. Esterification reactions can occur when an alcohol, such as methanol, reacts with an acid, such as citric acid, forming an ester and water. This reaction can decrease the solubility of the product and/or reduce the chelating effect of the treatment fluid, potentially compromising the effectiveness of the treatment fluid.

However, the presence of gluconic acid in the treatment fluid may help to mitigate this issue. Gluconic acid, being a sugar acid, contains alcohol groups that can enhance the solubility of the treatment fluid, even in the presence of methanol. This improved solubility may be particularly advantageous in cold climates, where the treatment fluid may otherwise be prone to freezing.

In some embodiments, the treatment fluid may be formulated to contain a specific concentration of methanol that is sufficient to lower the freeze point of the treatment fluid, but not so high as to promote esterification reactions. The methanol may be included in the treatment fluid in an amount of about 5 wt % to about 45 wt %, about 10 wt % to about 40 wt %, or about 15 wt % to about 38 wt % methanol, based on the total weight of the treatment fluid. The methanol may be included in the treatment fluid in an amount of about 15 wt %, based on the total weight of the treatment fluid. The methanol may be included in the treatment fluid in an amount of about 30 wt %, based on the total weight of the treatment fluid. This range of methanol concentration may provide a suitable balance between freeze point reduction and prevention or reduction of esterification reactions, thereby enhancing the overall performance and usability of the treatment fluid in a variety of operational contexts.

In summary, the present disclosure provides a treatment fluid and associated methods that offer potential advantages in the prevention and removal of polymer-polyvalent metal ion complex deposits in oil- and gas-field operations. The treatment fluid, comprising gluconic acid and hydrogen peroxide, and optionally citric acid, may be particularly effective in selectively binding to iron ions over other ions such as calcium, thereby offering a more targeted approach to preventing or removing these problematic deposits. Furthermore, the treatment fluid may exhibit improved solubility characteristics, particularly in cold climate formulations containing methanol, due to the presence of alcohol groups in gluconic acid.

In some aspects, gluconic acid may be produced biologically. Gluconic acid is a sugar acid derived from glucose, a simple sugar that is readily available in nature and can be produced from various sources, such as corn or other starch-rich materials. The biological production of gluconic acid may involve the enzymatic oxidation of glucose, where a glucose molecule is converted into gluconic acid through the action of an enzyme such as glucose oxidase. This enzymatic oxidation process may be carried out by various microorganisms, including bacteria, yeasts, and fungi, which naturally produce the necessary enzymes.

In some cases, the biological production of gluconic acid may be carried out in a fermentation process. In this process, a suitable microorganism may be cultured in a nutrient medium containing glucose. The microorganism may then convert the glucose into gluconic acid through enzymatic oxidation. The resulting gluconic acid may be recovered from the fermentation broth by various methods, such as precipitation, crystallization, or ion exchange.

In some embodiments, the gluconic acid produced through this biological process may be used directly in the formulation of the treatment fluid. Alternatively, the gluconic acid may be further processed or purified before being used in the treatment fluid. For example, the gluconic acid may be neutralized, concentrated, or dried to obtain a product that is suitable for use in the treatment fluid.

In summary, the biological production of gluconic acid provides a sustainable and potentially cost-effective method for obtaining this key component of the treatment fluid. The use of gluconic acid produced in this manner may contribute to the overall effectiveness and environmental compatibility of the treatment fluid in oil- and gas-field operations, water treatment, paper and pulp processing, disinfection, etc.

In some aspects, the method of formulating the treatment fluid may involve combining gluconic acid and hydrogen peroxide. This combination may be achieved by mixing the gluconic acid and hydrogen peroxide in a suitable container or vessel, such as a mixing tank or a reaction vessel. The mixing may be carried out under ambient conditions, or under controlled temperature and pressure conditions, depending on the specific requirements of the operation. The resulting mixture may then be further processed or treated to obtain the desired treatment fluid.

In some cases, the method of formulating the treatment fluid may further involve adding citric acid to the mixture of gluconic acid and hydrogen peroxide. The citric acid may be added in any suitable amount, depending on the specific needs of the operation. For example, in some cases, the citric acid may be added in an amount that is less than the amount of gluconic acid, in order to maintain the selective binding of the treatment fluid to iron ions. In other cases, the citric acid may be added in an amount that is equal to or greater than the amount of gluconic acid, depending on the desired properties of the treatment fluid.

In some embodiments, the treatment fluid may be formulated to have a specific concentration of gluconic acid and hydrogen peroxide. For example, the treatment fluid may comprise from about 1 ppm to about 40 wt % of the gluconic acid and hydrogen peroxide combined, based on the total weight of the treatment fluid. This range of concentration may provide a suitable balance between the chelating and oxidizing activities of the treatment fluid, while also ensuring sufficient solubility and stability of the treatment fluid in various operational contexts.

In some aspects, long-term stability testing of the gluconic acid formulations may be conducted to ensure the effectiveness and reliability of the treatment fluid over time. This testing may involve storing the treatment fluid under various conditions for a certain period of time, and then assessing the physical and chemical properties of the treatment fluid after the storage period. The testing may also involve evaluating the performance of the treatment fluid in preventing or removing polymer-polyvalent metal ion complex deposits, either in a laboratory setting or in actual field operations. The results of this testing may provide valuable information for optimizing the formulation of the treatment fluid and for predicting its performance in various operational contexts.

In one examples, corrosivity testing was conducted by retaining a mixture of gluconic acid, citric acid, and hydrogen peroxide in a carbon steel vessel for a three week duration. During the test period, no corrosion of the carbon steel vessel was observed.

The present method above can further include the step of adding a corrosion inhibitor so that the composition further contains the corrosion inhibitor. Any suitable corrosion inhibitor can be added. In some embodiments the corrosion inhibitor is a thiophosphate, pyrophosphate or oxygen phosphate as disclosed in U.S. Pat. No. 3,909,447; a tetrahydrothiazole phosphonic acid or phosphonate as disclosed in U.S. Pat. No. 4,443,609; blends of 2-amino-1-methoxypropane, sec-butylamine, n-butylamine, dipropylamine monoamylamine, isobutylamine; and 3-amino-1-methoxypropane as disclosed in U.S. Pat. No. 5,965,785; a sulfur and phosphorus-free aromatic compound substituted with nitrogen containing functional groups at the 5-, or 3, 5-position as disclosed in U.S. Pat. No. 6,593,278; a quaternary ammonium compound as disclosed in U.S. Pat. No. 8,177,962; or a phosphorus-based constituent and a sulfur-based constituent, nitrogen-based constituent, or combination thereof as disclosed in U.S. Patent Application Publication No. 20080001125, each incorporated herein by reference in their entireties.

In other embodiments, the corrosion inhibitor can be a phosphate ester, a derivative of the phosphate ester, a diacid, a derivative of the diacid, a quat amine, a derivative of the quat amine, an imidazoline, a derivative of the imidazoline, an alkyl pyridine, a derivative of the alkyl pyridine, a phosphonium salt, a derivative of the phosphonium salt, or a combination thereof.

In another embodiment, the corrosion inhibitor may include a neutralizing amine. Suitable neutralizing amines include morpholine, methoxypropylamine, ethyienediamine, monoethanolalmine, dimethylethanolamine diethylhydroxylamine, and hydrazine didrates.

In still another embodiment, the corrosion inhibitor may include a cationic surfactant comprising an ammonium halide. The ammonium halide may include any suitable types of ammonium halides. In embodiments, the ammonium halide includes alkyl ammonium halides, polyalkyl ammonium halides, benzyl triethyl ammonium halides or any combinations thereof. In embodiments, the cationic surfactant includes any combination or at least one of an alkyl trimethyl ammonium halide, alkyl triethyl ammonium halide, an alkyl dimethyl benzyl ammonium halide, and one or more imidazolinium halides.

In yet another embodiment, the corrosion inhibitor may include a phosphonate, including phosphonic acid and esters, such as tetrahydrothiazoles phosphonic acids or esters. In one embodiment, the phosphonate is an alkali metal (or functionally equivalent) salt of diethylene triamine penta(methylene phosphonic acid). Such phosphonates may be complete or partial salts and are preferably those of sodium and/or potassium, e.g. tripotassium diethylene triamine penta(methylene phosphonate), tetrasodium diethylene triamine penta(methylene phosphonate), etc. Mixtures of such phosphonates may also be employed if desired. Additional phosphorus-based compounds suitable for use, include thiophosphonic acid and the salts and alkyl, and aryl esters of the same.

In yet another embodiment, the corrosion inhibitor may be an alkali metal polyphosphate having the general formula Mₙ₊₂PₙO₃ₙ₊₁ where M is an alkali metal, including sodium and potassium or a cation functionally equivalent such as ammonium, n is an integer of at least 2, such as 2 to 20. Examples include, but are not limited to, di-, tri- and tetrasodium pyrophosphate, di-, tri- and tetra-potassium pyrophosphate, sodium tetrapolyphosphate, potassium hexametaphosphate, sodium hexametaphosphate, potassium tripolyphosphate and sodium tripolyphosphate. Such polyphosphates include complete and partial salts, as well as those having mixed cations, e.g. a sodium potassium tripolyphosphate. Mixtures of polyphosphates may also be used. It has been surprisingly found that the polyphosphates and phosphonates described above not only provide protection against corrosion, but may also allow the composition to exhibit a remarkably long shelf-life stability over an extended period of time across abroad a temperature range of from about 20° C. to about 70° C. For example, the composition may exhibit a shelf-life stability of at least 1 week, or at least 2 weeks, or at least 3 weeks, or at least 1 month, or at least 6 weeks, or at least 2 months, or at least 10 weeks, or at least 3 months at a temperature ranging rom about 20° C. to about 70° C. Thus, in another embodiment, there is provided a method of stabilizing the composition for an extended period of time across a temperature range of from about 20° to about 70° C. including the step of adding a corrosion inhibitor comprising a polyphosphate or phosphonate to the composition. In still another embodiment, there is provided the composition of the present disclosure that further includes a stabilizing effective amount of a corrosion inhibitor comprising a polyphosphate or phosphonate, the stabilizing effective amount being any amount which will effectively retard or prevent degradation of the composition for an extended period of time at a temperature range from about 20° C. to about 70° C. According to another embodiment, there is provided a method for storing a peroxy gluconic acid (and optionally, percitric acid) containing composition, which method includes storing the above compositions, wherein the composition retains at least about 80% of the peroxy gluconic acid (and optionally, percitric acid) activity after storage for any suitable time under any suitable conditions, e.g., retaining at least about 80% of the percitric acid activity after storage of about 30 days at a temperature ranging between about 20° C. to about 70° C. Preferably, the present compositions retain at least about 85%, 90% or higher of the peroxy gluconic acid (and optionally percitric acid) activity after storage of about 30 days at a temperature ranging between about 20° C. to about 70° C.

The corrosion inhibitor can be added at any suitable time. In some embodiments, the corrosion inhibitor can be added before the gluconic acid (and optionally, citric acid) is contacted with the hydrogen peroxide. In other embodiments, the corrosion inhibitor can be added concurrently when the gluconic acid (and optionally, citric acid) is contacted with the hydrogen peroxide. In still other embodiments, the corrosion inhibitor can be added after the gluconic acid (and optionally, citric acid) has been contacted with the hydrogen peroxide.

In an embodiment, the corrosion inhibitor may be present in the composition in an amount of between about 1 ppm to 100,000 ppm. In a further embodiment, the corrosion inhibitor is present in the composition in an amount of at least about 0.0001 wt. % (w/w) to about 20 wt. %, such as at least about 0.0001 wt. % to about 10 wt. %, or at least about 0.0001 wt. % to about 5 wt. %, or at least about 0.0001 wt. % to about 1 wt. %, or at least about 0.0001 wt. % to about 0.1 wt. %, or at least about 0.0001 wt. % to about 0.05 wt. %, based on the total weight of the composition.

The present method above can be used to generate peroxy gluconic acid in any suitable manner or at any suitable location. For example, in some embodiments, the present method above can be used to generate peroxy gluconic acid in situ for the application of the formed peroxy gluconic acid. In other non-limiting embodiments, a percarbonate, persulfate, perborate or other oxidizer is used to generate hydrogen peroxide in situ upon reaction with water (or an oxygen-containing gas is supplied to an electrolysis unit to produce peroxide species) which can then react with gluconic acid to form peroxy gluconic acid.

The present method above can be used to generate percitric acid in any suitable manner or at any suitable location. For example, in some embodiments, the present method above can be used to generate percitric acid in situ for the application of the formed percitric acid. In other non-limiting embodiments, a percarbonate, persulfate, perborate or other oxidizer is used to generate hydrogen peroxide in situ upon reaction with water (or an oxygen-containing gas is supplied to an electrolysis unit to produce peroxide species) which can then react with citric acid to form percitric acid.

The composition described above may be combined with one or more chemical additives to form a treatment fluid. Examples of chemical additives include, but are not limited to, polymers, wetting agents, organophilic clays, oxygen scavengers, proppants, biocides, weighting agents, foaming agents, surfactants, dispersants, interfacial tension reducers, solvents, thinning agents, stabilizing agents/pH buffers, scale inhibitors and mixtures thereof.

The polymer can be any polymer used in an oil- and gas-field operation, water treatment, paper and pulp processing, disinfection, etc. for modifying the viscosity, stability, or other property of the treatment fluid. For example, the polymer may be a polysaccharide, such as a galactomannan polymer (for example, guar gum), a derivatized galactomannan polymer, starch, xanthan gum, a derivatized cellulose (for example, hydroxy cellulose or hydroxyalkyl cellulose), a polyvinyl alcohol polymer, or a synthetic polymer that is the product of a polymerization reaction comprising one or more monomers selected from the group consisting of vinyl pyrrolidone, 2-acrylamido-2-methylpropanesulfonic acid, acrylic acid, methacrylic acid, styrene sulfonic acid, acrylamide, and other monomers currently used for viscosity-modifying polymers.

As used herein, the terms “polymer,” “polymers,” “polymeric,” and similar terms are used in their ordinary sense as understood by one skilled in the art, and thus may be used herein to refer to or describe a large molecule (or group of such molecules) that contains recurring units. Polymers may be formed in various ways, including by polymerizing monomers and/or by chemically modifying one or more recurring units of a precursor polymer. A polymer may be a “homopolymer” comprising substantially identical recurring units formed by, for example, polymerizing a particular monomer. A polymer may also be a “copolymer” comprising two or more different recurring units formed by, for example, copolymerizing two or more different monomers, and/or by chemically modifying one or more recurring units of a precursor polymer. The term “terpolymer” may be used herein to refer to polymers containing three or more different recurring units. The term “polymer” as used herein is intended to include both the acid form of the polymer as well as its salts.

In some cases, the polymer can be a water-soluble polymer. In certain cases, the polymer can comprise a biopolymer, such as a polysaccharide. For example, the polysaccharide can be chosen from cellulose, galactomannan, derivatives thereof, and combinations thereof. Other examples include xanthan, starch, derivatives thereof, and combinations thereof. In some examples, the polysaccharide can comprise guar gum or a derivative thereof. Examples of guar derivatives include hydroxypropyl guar (UPG), carboxymethyl guar (CMG), and carboxymethyl hydroxypropyl guar (CMHPG). In some examples, the polysaccharide can comprise cellulose or a derivative thereof. Examples of cellulose derivatives include hydroxyethyl cellulose (HEC),

In certain embodiments, the polymer can comprise a carbon-carbon backbone. For example, the polymer can comprise a synthetic polymer, such as a (co)polymer derived from one or more ethylenically unsaturated monomers. Examples of ethylenically unsaturated monomers include, for example, acrylamide derivatives such as, for example, N-methyl(meth)acrylamide, N,N′-dimethyl(meth)acrylamide, and N-methylolacrylamide, N-vinyl derivatives such as N-vinylformamide, N-vinylacetamide, N-vinylpyrrolidone or N-vinylcaprolactam, and vinyl esters, such as vinyl formate or vinyl acetate, vinyl aromatic monomers (for example, styrene), acid-containing monomers (for example, monomers comprising —COOH groups, such as acrylic acid or methacrylic acid), and monomers comprising sulfonic acid groups, such as vinylsulfonic acid, allylsulfonic acid, or 2-acrylamido-2-methylpropanesulfonic acid), and (meth)acrylate monomers. Examples of such polymers include polyacrylic acid, polyacrylamide (PAM), partially hydrolyzed polyacrylamide (HPAM), acrylamide-methyl-propane sulfonate (AMPS), copolymers thereof, and combinations thereof.

In other embodiments, the polymer can comprise a polyether. Examples of polyethers include poly(ethylene oxide) (also referred to as polyethylene glycol (PEG)), poly(propylene oxide) (also referred to as polypropylene glycol (PPG)), polytetramethylene glycol (PTMG), copolymers thereof, and combinations thereof.

In certain embodiments, the polymer can comprise a polyacrylamide. The polyacrylamide can comprise a polyacrylamide (co)polymer derived from acrylamide, N-substituted hydrosoluble derivatives of acrylamide, hydrosoluble N-vinylamides, and combinations thereof.

In other embodiments, the polymer may either be linear (non-crosslinked) or crosslinked, for example, using cross-linking agents such as borate or zirconate or titanate in the case of polysaccharides like guar, or other known crosslinkers in the case of synthetic polymers and copolymers like acrylamide-derived polymers and copolymers.

In one embodiment, the polymer may be present in an amount of at least about 0.001 wt. % to about 10 wt. %, preferably at least about 0.01 wt. % to about 5 wt. %, preferably at least about 0.01 wt. % to about 3 wt. %, at least about 0.01 wt. % to about 2 wt. %, preferably at least about 0.01 wt. % to about 1 wt. %, or preferably at least about 0.01 wt. % to about 0.5 wt. %, based on the total weight of the treatment fluid.

Exemplary wetting agents that may be suitable for use in the treatment fluids of the disclosed subject matter include, but are not limited to, crude tall oil, oxidized crude tall oil, organic phosphate esters, modified imidazolines and amidoamines, oleic acid based wetting agents, alkyl aromatic sulfates and sulfonates, and the like, and combinations or derivatives of these.

Examples of organophilic clays include, but are not limited to, a modified phyllosilicate (sheet silicate), more preferably a clay mineral modified with an organic cation (for example, a quaternary ammonium cation). For example, the organophilic clay may be a sheet silicate/clay mineral ion-exchanged with an organic cation. Suitable sheet silicates/clay minerals to be modified may include, but are not limited to, a kaolin, a smectite (for example, bentonite, montmorillonite, hectorite, laponite, biedellite, saponite, stevensite), an illite, a pyrophyilite, an attapugite/palygorskite, a sepiolite, or a mixture thereof.

Exemplary oxygen scavengers include, but are not limited to, sulfites, and bisulfites. Other oxygen scavengers are also possible and will be known to those skilled in the art.

Examples of proppants include, but are not limited to, sand, glass beads, walnut hulls, metal shot, resin-coated sands, intermediate strength ceramics, sintered bauxite, resin-coated ceramic proppants, plastic beads, polystyrene beads, thermoplastic particulates, thermoplastic resins, thermoplastic composites, thermoplastic aggregates containing a binder, synthetic organic particles including nylon pellets and ceramics, ground or crushed shells of nuts, resin-coated ground or crushed shells of nuts, ground or crushed seed shells, resin coated ground or crushed seed shells, processed wood materials, porous particulate materials, and combinations comprising at least one of the foregoing. Ground or crushed shells of nuts can comprise shells of pecan, almond, ivory nut, brazil nut, macademia nut, or combinations comprising at least one of the foregoing. Ground or crushed seed shells can include fruit pits, and can comprise seeds of fruits including plum, peach, cherry, apricot, and combinations comprising at least one of the foregoing. Ground or crushed seed shells can further comprise seed shells of other plants including maize, for example corn cobs and corn kernels. Processed wood materials can comprise those derived from woods including oak, hickory, walnut, poplar, and mahogany, and includes such woods that have been processed by any means that is generally known including grinding, chipping, or other forms of particulization. A porous particulate material can be any porous ceramic or porous organic polymeric material, and can be natural or synthetic. The porous particulate material can further be treated with a coating material, a penetrating material, or modified by glazing. Proppants are typically used in concentrations between about 1 to 8 lbs. per gallon of the treatment fluid, although higher or lower concentrations may also be used as desired.

Non-limiting examples of biocides include didecyl dimethyl ammonium chloride, gluteral, Dazomet, bronopol, tributyl tetradecyl phosphonium chloride, tetrakis (hydroxymethyl) phosphonium sulfate, glutaraldehyde, sodium hypochlorite, and sodium hydroxide. Other biocides are also possible and will be known to those skilled in the art.

The biocide may be employed in amounts sufficient to provide antimicrobial efficacy, as may vary depending upon the water source in need of treatment and the contaminants therein. Such biocides may be present in an amount of at least about 0.1 wt. % to about 20 wt. %, preferably at least about 0.1 wt. % to about 10 wt. %, more preferably from about 0.1 wt. % to about 5 wt. %, based on the total weight of the treatment fluid.

Examples of weighting agents include, but are not limited to, barite, hematite, calcium carbonate, siderite, and limonite. Exemplary examples of foaming agents include, without limitation, silicone foaming agents such as tetra(trimethylsiloxy)silane or polysiloxanes, fluorinated oligomeric or polymeric foaming agents such as fluorinated methacrylic copolymer or other fluorosurfactants.

Examples of surfactants that may be useful include anionic surfactants, cationic surfactants, nonionic surfactants, amphoteric surfactants (for example, zwitterionic surfactants), and combinations thereof. Many of each type of surfactant are widely available to one skilled in the art. These include fluorochemical, silicone and hydrocarbon-based surfactants.

Examples of anionic surfactants include, but are not limited to, alkali metal and (alkyl)ammonium salts of: alkyl sulfates and sulfonates such as sodium dodecyl sulfate and potassium dodecanesulfonate; sulfates of polyethoxylated derivatives of straight or branched chain aliphatic alcohols and carboxylic acids; alkylbenzenesulfonates, alkylnaphthalenesulfonates and sulfates (for example, sodium laurylbenzenesulfonate); ethoxylated and polyethoxylated alkyl and aralkyl alcohol carboxylates; glycinates (for example, alkyl sarcosinates and alkyl glycinates); sulfosuccinates including dialkyl sulfosuccinates; isethionate derivatives; N-acyltaurine derivatives (for example, sodium N-methyl-N-oleyl taurate); and alkyl phosphate mono- or di-esters (for example, ethoxylated dodecyl alcohol phosphate ester, sodium salt.

Examples of cationic surfactants include, but are not limited to, alkylammonium salts having the formula CᵣH₂ᵣ₊₁N(CH₃)₃X, where X is OH, Cl, Br, HSO₄ or a combination of OH and Cl, and where r is an integer from 8 to 22, and the formula CₐHₐ₊₁N(C₂H₅)₃X, where a is an integer from 12 to 18; gemini surfactants, for example, those having the formula: [C₁₆H₃₃N(CH₃)₂CₜH₂ₜ₊₁]X, wherein t is an integer from 2 to 12 and X is OH, Cl, Br, HSO₄ or a combination of OH and Cl; aralkylammonium salts (for example, benzalkonium salts); and cetylethylpiperidinium salts, for example, C₁₆H₃₃N(C₂H₅)(C₅H₁₀)X, wherein X is OH, Cl, Br, HSO₄ or a combination of OH and Cl.

Examples of amphoteric surfactants include, but are not limited to, alkyldimethyl amine oxides, alkylcarboxamidoalkylenedimethyl amine oxides, aminopropionates, sulfobetaines, alkyl betaines, alkylamidobetaines, dihydroxyethyl glycinates, imidazoline acetates, imidazoline propionates, ammonium carboxylate and ammonium sulfonate amphoterics and imidazoline sulfonates.

Examples of hydrocarbon nonionic surfactants include, but are not limited to, polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ethers, polyoxyethylene cetyl ethers, polyoxyethylene stearyl ethers, polyoxyethylene oleyl ether, polyoxyethylene octylphenyl ethers, polyoxyethylene nonylphenyl ethers, polyethylene glycol laurates, polyethylene glycol stearates, polyethylene glycol distearates, polyethylene glycol oleates, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurates, polyoxyethylene sorbitan stearates, polyoxyethylene sorbitan oleates, polyoxyethylene laurylamines, polyoxyethylene laurylamides, laurylamine acetate, ethoxylated tetramethyldecynediol, fluoroaliphatic polymeric ester, and polyether-polysiloxane copolymers.

In some embodiments, the surfactant may present in an amount of about 0.005 wt. % to about 15 wt. %, or about 0.01 wt. % to about 10 wt. % or about 0.01 wt. % to about 5 wt. %, or about 0.01 wt. % to about 2 wt. % or about 0.5 wt. % to about 1.0 wt. %, based on the total weight of the treatment fluid.

Examples of dispersants include, but are not limited to, sulfonated styrene maleic anhydride copolymer, sulfonated vinyl toluene maleic anhydride copolymer, sodium naphthalene sulfonate condensed with formaldehyde, sulfonated acetone condensed with formaldehyde, lignosulfonates (e.g., modified sodium lignosulfonate), allyloxybenzene sulfonate, allyl sulfonate and non-ionic monomers, and interpolymers of acrylic acid.

Examples of solvents include, but are not limited to, water, flowback fluids, ethylene glycol monobutyl ether, methanol, isopropanol or mixtures thereof. For example, in one embodiment methanol may be added to the treatment fluid to prevent its freezing in cold climates and in such embodiments may be added in amounts that can range from about 1 wt. % to about 15 wt %, based on the total weight of the treatment fluid. In some embodiments, the amount of solvent can be about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, or about 15 wt. %, based on the total weight of the treatment fluid. In other embodiments, the solvent may present in an amount of between about 0.1 wt. % to about 50 wt. % or about 0.1 wt. % to about 40 wt. % or about 0.1 wt. % to about 30 wt. % or about 0.1 wt. % to about 20 wt. % or about 0.1 wt. % to about 10 wt. % or about 0.1 to about 5 wt. %, based on the total weight of the treatment fluid.

In some aspects, the treatment fluid may further comprise methanol. The methanol may be included in the treatment fluid in an amount of about 5 wt % to about 45 wt %, about 10 wt % to about 40 wt %, or about 15 wt % to about 38 wt %, based on the total weight of the treatment fluid. The methanol may be included in the treatment fluid in an amount of about 15 wt %, based on the total weight of the treatment fluid. The methanol may be included in the treatment fluid in an amount of about 30 wt %, based on the total weight of the treatment fluid. The inclusion of methanol in the treatment fluid may be advantageous in certain operational contexts, particularly in cold climates. For instance, the methanol may help to lower the freeze point of the treatment fluid, thereby enhancing its usability and effectiveness in cold weather conditions. In some cases, the treatment fluid may have a freeze point of about 0° F. to about −40° F., depending on the specific concentration of methanol used. In some examples, the methanol may be included in the treatment fluid in an amount of about 15 wt %, based on the total weight of the treatment fluid to obtain a treatment fluid having a freeze point of about 0° F. In some examples, the methanol may be included in the treatment fluid in an amount of about 30 wt %, based on the total weight of the treatment fluid to obtain a treatment fluid having a freeze point of about −40° F.

In some embodiments, the treatment fluid may be formulated by adding methanol to a mixture of gluconic acid and hydrogen peroxide, and optionally citric acid. The specific amount of methanol added may be selected based on various factors, such as the desired freeze point of the treatment fluid and the specific operational context. For example, a higher concentration of methanol may be used to achieve a lower freeze point, which may be particularly advantageous in operations conducted in extremely cold climates.

In some cases, the gluconic acid present in the treatment fluid may help to improve the solubility of the treatment fluid in cold climate formulations containing methanol. The presence of alcohol groups in gluconic acid may enhance the solubility of the treatment fluid, thereby facilitating its use in a wider range of operational contexts and conditions. This may be particularly advantageous in operations conducted in cold climates, where the solubility of the treatment fluid may otherwise be compromised due to the low temperatures.

Non-limiting examples of thinning agents suitable for use in the present disclosure include, but are not limited to, alkoxylated fatty alcohol, and non-ionic surfactants which comprise reaction products of ethylene oxide, propylene oxide and/or butylene oxide with C₁₀-C₂₂ carboxylic acids or C₁₀-C₂₂ carboxylic acid derivatives. Thinning agents suitable for use with the present disclosure are described in more detail in U.S. Pat. Nos. 7,435,706 and 7,638,466, each of which is incorporated by reference herein in their entirety.

Stabilizing agents may be added to equilibrium acid (peroxy gluconic acid and/or percitric acid)-containing compositions to stabilize the acid and hydrogen peroxide and prevent the decomposition of these constituents within the compositions. Various embodiments of the present disclosure do not require the use of at least one or more of such stabilizing agents. Examples of stabilizing agents may include couplers, hydrotropes, acid catalysts and the like that are conventionally used in equilibrium acid-containing compositions to stabilize and improve shelf life of the composition.

Further examples of stabilizing agents include chelating agents or sequestrants. Such sequestrants include, but are not limited to, organic chelating compounds that sequester metal ions in solution, particularly transition metal ions. Such sequestrants include organic amino- or hydroxy-polyphosphonic acid complexing agents (either in acid or soluble salt forms), carboxylic acids (e.g., polymeric poly carboxylate), hydroxy carboxylic acids, aminocarboxylic acids, or heterocyclic carboxylic acids, e.g., pyridine-2,6-dicarboxylic acid (dipicolinic acid). Dipicolinic acid, 1-hydroxy ethylidene 1,1-diphosphonic acid (CH₃C(PO₃H₂)₂OH) (HEDP) are further example of stabilizing agents.

Additional examples of stabilizing agents commonly used in equilibrium chemistry to stabilize the acid (peroxy gluconic acid and/or percitric acid) and hydrogen peroxide and/or prevent the premature oxidation of the composition include phosphonic acid or phosphonate salt. Phosphonic acids and phosphonate salts include HEDP; ethylenediamine tetrakis methylenephosphonic acid (EDTMP); diethylenetriamine pentakis methylenephosphonic acid (DTPMP); cyclohexane-1,2-tetramethylene phosphonic acid; amino [tri(methylene phosphonic acid)]; (ethylene diamine[tetra methylene-phosphonic acid)]; 2-phosphene butane- 1,2,4-tricarboxylic acid; or salts thereof, such as the alkali metal salts, ammonium salts, or alkyloyl amine salts, such as mono, di, or tetra-ethanolamine salts; picolinic, dipicolinic acid or mixtures thereof. In some embodiments, organic phosphonates, for example, HEDP are well known as used stabilizing agents.

Examples of pH buffers include, but are not limited to, potassium hydroxide, sodium hydroxide, phosphate buffer, bicarbonate buffer and Tris buffer. However, it is to be understood that the treatment fluid is effective even if its pH is raised and the conjugate bases of the peracids are formed. In some examples, the pH buffer, e.g., potassium hydroxide or sodium hydroxide, may react with a carboxyl group of a constituent of the treatment fluid, and the reaction product may act as the pH buffer.

In one embodiment, the stabilizing agent/pH buffer is present in amounts from about 0.001 wt. % to about 30 wt. %, or about 0.001 wt. % to about 20 wt. %, or about 0.001 wt. % to about 10 wt. %, or about 0.01 wt. % to about 1 wt. %, based on the total weight of the treatment fluid.

Examples of scale inhibitors include, but are not limited to, phosphates, phosphate esters and the like. The scale inhibitor may present in an amount ranging between about 0.0001 wt. % to about 10 wt. %, or at least about 0.0001 wt. % to about 1 wt. %, preferably at least about 0.0001 wt. % to about 0.1 wt. %, preferably at least about 0.0001 wt. % to about 0.05 wt. %, based on the total weight of the treatment fluid.

The above-described treatment fluids may be included in various methods performed during oil- and gas-field operations, water treatment, paper and pulp processing, disinfection, etc. The treatment fluids may be used at any temperatures, such as at a temperature of between about −40° F. and about 400° F. (about −40° C. and about 204° C.). Preferably, the treatment fluids are used at a temperature where they best achieve the desired effects, which may be a temperature of between 77° F. and about 300° F. (about 25° and about 149° C.).

The treatment fluids may be used in oil- and gas-field operations. In some embodiments, the use of the treatment fluids may be at a pressure between atmospheric pressure and fracture pressure, where the fracture pressure is defined as the pressure above which injection of fluids will cause the formation to fracture hydraulically. In addition, the treatment fluids may be used in any subterranean formation. For example, the subterranean formation may be a sandstone or a carbonate formation. Sandstone formations contain siliceous materials, like quartz, as the major constituent and may contain various amounts of clays (aluminosilicates such as kaolinite or illite) or alkaline aluminosilicates such as feldspars, and zeolites, as well as carbonates (calcite, dolomite, ankerite) and iron-based minerals (hematite and pyrite). Carbonate formations contain calcium carbonates (dolomite and calcite) as the major constituent.

As discussed above, residual polymers can present difficulties in a variety of fields, including in various oil- and gas-field operations. Some polymers, especially polymers with carbon-carbon backbones, are particularly recalcitrant to traditional remediation techniques.

Provided herein are improved methods for degrading polymer systems using the treatment fluids of the present disclosure. These treatment fluids and methods may be used to effectively degrade a range of polymers commonly used in oil- and gas-field operations, water treatment, paper and pulp processing, disinfection, etc., including polymers with carbon-carbon backbones such as polyacrylamides (and the polymers described above). Further, these treatment fluids and methods may simultaneously degrade other organic molecules and pollutants associated with oil- and gas-operations. Finally, these treatment fluids and methods employ components that are more environmentally-friendly than conventional methods and components used in polymer breaking processes.

In some embodiments, the methods described herein can employ a treatment fluid containing an acid (peroxy gluconic acid and/or percitric acid) to degrade a polymer (for example, as part of a hydraulic fracturing operation and/or as part of a remediation operation) or remove contaminants in various applications such as oil- and gas-field operations, water treatment processes, paper and pulp processing, or disinfection activities. The methods can be adapted for degrading a polymer within a substrate, such as subterranean formation, the subterranean formation being in fluid communication with a wellbore. The methods can comprise (a) introducing a treatment fluid comprising peroxy gluconic acid (and optionally, percitric acid) into the substrate (e.g., wellbore, water treatment infrastructure, processing pipeline, or disinfection surface); and (b) contacting the substrate (e.g., subterranean formation) with the treatment fluid for an effective amount of time to degrade the polymer. The step of introducing can include injecting. The injecting can be performed in a single injecting event, multiple injecting events, or as a continuous injecting process. The term “injecting” as used herein, refers to any mechanical device like a centrifugal pump, a piston pump, gear pump or any other device, commonly known in the art, for pressurizing and transporting a fluid from a low-pressure environment into a higher pressure environment. The term “contacting” means direct or indirect binding or interaction between two or more entities (e.g., between the subterranean formation and treatment fluid).

In some embodiments, the amount of peroxy gluconic acid present in the treatment fluid may be from about 0.1 ppm to about 100,000 ppm, based on the total weight of the treatment fluid, or any range therein. For example, the amount of peroxy gluconic acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of peroxy gluconic acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

In some embodiments, the amount of percitric acid present in the treatment fluid may be from about 0.1 ppm to about 100,000 ppm, based on the total weight of the treatment fluid, or any range therein. For example, the amount of percitric acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of percitric acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

In some embodiments, the total amount of acid present (e.g., peroxy gluconic acid and/or percitric acid) in the treatment fluid may be from about 0.1 ppm to about 100,000 ppm, based on the total weight of the treatment fluid, or any range therein. For example, the amount of percitric acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of acid (e.g., peroxy gluconic acid and/or percitric acid) may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

In some embodiments, the contacting step in the present methods can last for any suitable amount of time. In some embodiments, the contacting step can last for at least about 10 seconds. For example, the contacting step can last for at least about 30 seconds, 40 seconds, 50 seconds, 1 minute, 2 minutes, 3 minutes, 4 minutes, 5 minutes, 10 minutes, 15 minutes, 20 minutes, 25 minutes, 30 minutes, 40 minutes, 50 minutes, 1 hour, 2 hours, 3 hours, 4 hours, 5 hours, 6 hours, 7 hours, 8 hours 9 hours, 10 hours, 16 hours, 1 day, 3 days, 1 week, or until degradation or exhaustion of the chemistry.

In some embodiments, the polymer can be deposited on a substrate, such as a surface within the subterranean formation. For example, the polymer can comprise a filter cake formed on a surface within the subterranean formation from a fluid used for drilling or fracturing. In certain embodiments, the polymer can comprise a polyacrylamide-containing filter cake in a well that is formed during drilling or completion of the well.

Also provided are methods of treating a substrate, such as a subterranean formation, a water treatment system, a paper and pulp processing pipeline, or a disinfection surface, that comprise (a) introducing or injecting a first treatment fluid comprising a polymer into the substrate; and (b) reducing or breaking the viscosity of or disrupting the polymer structure in the first treatment fluid by fragmenting the polymer, wherein the polymer is fragmented by introducing or injecting a second treatment fluid comprising acid (peroxy gluconic acid and/or percitric acid) into the substrate (e.g., subterranean formation). By way of example, the second treatment fluid may interact with the polymer to reduce the viscosity of the first treatment fluid forming a reduced viscosity treatment fluid. The reduced viscosity treatment fluid may then be recovered from the substrate (e.g., subterranean formation).

Also provided are methods of slickwater fracturing a subterranean formation that comprise (a) injecting a first treatment fluid comprising a polymer, such as polyacrylamide, into the subterranean formation at a pressure sufficient to create or enlarge a fracture in the subterranean formation; and (b) breaking the viscosity of the first treatment fluid by fragmenting the polyacrylamide, wherein the polyacrylamide is fragmented by injecting a second treatment fluid comprising an acid (peroxy gluconic acid and/or percitric acid) into the subterranean formation.

For example, in some embodiments, the above methods of breaking may reduce the viscosity of the first treatment fluid to a viscosity that is less than the viscosity of the first treatment fluid prior to injecting the second treatment fluid. For example, the breaking may reduce the viscosity of the first treatment fluid to a viscosity less than about 100,000 cP, or less than about 50,000 cP, or less than about 10,000 cP, or less than about 1000 cP. In other embodiments the breaking may reduce the viscosity of the first treatment fluid to less than about 500 cP, less than about 100 cP, less than about 50 cP, less than about 40 cP, less than about 30 cP, less than about 20 cP, less than about 15 cP, less than about 10 cP, less than about 5 cP, less than about 1 cP.

The amount of peroxy gluconic acid present in the second treatment fluid may be from about 0.1 ppm to about 100,000 ppm, based on the total weight of the treatment fluid, or any range therein. For example, the amount of peroxy gluconic acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of peroxy gluconic acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

The amount of percitric acid present in the second treatment fluid may be from about 0.1 ppm to about 100,000 ppm, based on the total weight of the treatment fluid, or any range therein. For example, the amount of percitric acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of percitric acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

The total amount of acid (e.g., peroxy gluconic acid and/or percitric acid) present in the second treatment fluid may be from about 0.1 ppm to about 100,000 ppm, based on the total weight of the treatment fluid, or any range therein. For example, the total amount of acid (e.g., peroxy gluconic acid and/or percitric acid) may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the total amount of acid (e.g., peroxy gluconic acid and/or percitric acid) may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

Also provided herein are methods for degrading polymers within fluid handling systems associated with various industrial and operational processes, including oil- and gas-field operations, water treatment processes, paper and pulp processing, and disinfection activities. For example, the methods described herein can be used to degrade, disintegrate, decompose and/or remove accumulated polymer deposited within one or more components of an fluid handling systems (which may be above-ground), including fluid lines, filters, membranes, separator tanks, settling tanks, processing pipelines, storage tanks, or other components that come into contact with polymer-containing fluids.

Methods for degrading, disintegrating or decomposing polymers within fluid handling systems can comprise contacting one or more of the components of the fluid handling system with a treatment fluid comprising an acid (peroxy gluconic acid and/or percitric acid) for an effective amount of time to fragment the polymer. In some embodiments, this can comprise flowing the treatment fluid through the components of the fluid handling system.

In some embodiments, methods can further comprise washing or flushing the components of the fluid handling system with water or a compatible fluid (e.g., flowback fluid, recycled water, or clean process water) to remove the degraded polymer from at least one component and, in some embodiments, from the fluid handling system.

The amount of peroxy gluconic acid present in the treatment fluid may range from about 0.1 ppm to about 100,000 ppm, based on the total weight of the treatment fluid, or any range therein. For example, the amount of peroxy gluconic acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of peroxy gluconic acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

The amount of percitric acid present in the treatment fluid may range from about 0.1 ppm to about 100,000 ppm, based on the total weight of the treatment fluid, or any range therein. For example, the amount of percitric acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of percitric acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

The total amount of acid (e.g., peroxy gluconic acid and/or percitric acid) present in the treatment fluid may range from about 0.1 ppm to about 100,000 ppm, based on the total weight of the treatment fluid, or any range therein. For example, the total amount of acid (e.g., peroxy gluconic acid and/or percitric acid) may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the total amount of acid (e.g., peroxy gluconic acid and/or percitric acid) may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treatment fluid.

The contacting step can last for any suitable amount of time. In some embodiments, the contacting step can last for at least about 10 seconds. For example, the contacting step can last for at least about 30 seconds, 40 seconds, 50 seconds, 1 minute, 2 minutes, 3 minutes, 4 minutes, 5 minutes, 10 minutes, 15 minutes, 20 minutes, 25 minutes, 30 minutes, 40 minutes, 50 minutes, 1 hour, 2 hours, 3 hours, 4 hours, 5 hours, 6 hours, 7 hours, 8 hours 9 hours, 10 hours, 16 hours, 1 day, 3 days, 1 week or longer.

According to another embodiment, the present disclosure relates to methods of preventing polymer deposition during various industrial or operational processes, including oil- and gas-field operations, water treatment, paper and pulp processing, and disinfection activities. Some embodiments disclosed herein relate to preventing the formation of such polymer deposits on oilfield equipment, in a wellbore, and on or within the subterranean formation during oil- and gas-field operations. The inventors have advantageously found that a treatment fluid containing an acid (peroxy gluconic acid and/or percitric acid) may prevent the buildup of polymer deposits on substrates (e.g., in the wellbore, on or within the subterranean formation, or on downhole equipment). The method generally includes introducing the treatment fluid containing an acid (peroxy gluconic acid and/or percitric acid) into substrate (e.g., a wellbore in fluid communication with the subterranean formation containing a polymer during an oil- and gas-field operation) and contacting the polymer with the treatment fluid for an effective amount of time to prevent the polymer from depositing on or within the substrate (e.g., subterranean formation).

Polymers found in treatment fluids are also capable of complexing with polyvalent metal ions (e.g., found in the wellbore and within the subterranean formation), forming polymer scale (e.g., when drilling through formations of that type and during completions). The issue can arise during oil- and gas-field operations, as well as in water treatment systems, paper and pulp processing pipelines, or disinfection activities, where treatment fluids interact with polyvalent metal ions in the system. According to another embodiment, it has been found that when an acid (peroxy gluconic acid and/or percitric acid) is present in the treatment fluid, it may prevent the buildup of polymer scale in the substrate (e.g., wellbore, on downhole equipment, or on or within the subterranean formation itself). For example, an acid (peroxy gluconic acid and/or percitric acid) may be added to treatment fluids generally used in the normal course of drilling or oil recovery, water treatment, paper and pulp processing, or cleaning operations. This improved treatment fluid may then be used to prevent the buildup of polymer scale on equipment, within pipelines, tanks, surfaces, etc., ensuring efficient operation across various industries. For example, this improved treatment fluid may then be used in drilling or in oil recovery operations to prevent the buildup of polymer scale in the wellbore, on the downhole equipment, and on the earthen formation. The present disclosure addresses any polymer scale that is or may be induced by the interaction of polyvalent metal ions and polymer, regardless of the source of the polyvalent metal ions.

In still other embodiments, it has been found that the presence of peroxy gluconic acid and/or percitric acid in a treatment fluid used as a remediation fluid may remove polymer scale from the equipment, pipelines, tanks, surfaces, wellbore, on downhole equipment, or on or within the subterranean formation itself. For example, after polymer scale has been observed on the equipment, within pipelines, or thought to exist in the system or on the subterranean formation, the remediation fluid of the present disclosure may be introduced (e.g., in downhole). The remediation fluid may then remove the polyvalent ions from the crosslinked polymer, allowing the polymer to return to its fluid, un-crosslinked state. The polymer may then be recycled into a treatment fluid for circulation or other use in the system or process (e.g., in the wellbore). Alternatively, the remediation fluid may be used to remove polymer scale from equipment across industries, such as oil and gas operations, water treatment facilities, paper and pulp processing systems, or disinfection processes.

Polyvalent metal cations which may crosslink the polymer may stem from minerals naturally present in the environment (e.g., subterranean formations), from metallic substances in equipment (e.g., oilfield), or from base fluids used in formulating treatment fluids (e.g., brines, process water, or cleaning solutions). Nonlimiting examples of such metal cations include calcium, magnesium, strontium, boron, cobalt, copper, zirconium, titanium, aluminum, iron, chromium, hafnium, and the like. For instance, Fe(II)/Fe(III) cations are dissolved from iron-containing minerals and solids in the downhole environment, water treatment systems, paper and pulp processing equipment, or disinfection systems, where they may crosslink with the polymer to form scale. As a result, the polymer crosslinked with Fe(II)/Fe(III) cations may form an insoluble solid deposition or scale on the formation, equipment (e.g., oilfield), pipelines, membranes, or other system components.

The result of this crosslinking is polymer immobilization and plugging of the substrate (e.g., formation) due to a gelation mechanism. Heavy metal ions such as Cr³⁺, Al³⁺, Fe²⁺, Ca²⁺ and Fe³⁺ are well known to cause gelation of the polymer. There are also other ions which may complex with the polymer in certain pH intervals. Polymer gelation is thought to occur via the carboxylic groups, and the mechanism of gelation does not appear to selectively favor any polyvalent ion over others. The crosslinking reaction is thought to be a ligand exchange reaction where water molecules coordinated to the heavy metal ion are exchanged for the carboxylic groups of the polymer. The polyvalent heavy metal ion may complex to several carboxylic groups of the polymer backbone causing the polymer chain to crosslink with itself, or with other polymer chains forming an insoluble scale or gel.

In oil and gas operations, polymer gelation can plug formations, reduce hydrocarbon production, and create deposits in wellbores, casing perforations, tubing strings, and equipment. When used in flooding, oil production is thus reduced because the polymer cannot readily migrate through the rock formation. Similarly, scale deposits can also result in plugging of wellbores, well casing perforations and tubing strings, as well as sticking of downhole safety valves, downhole pumps and other downhole and surface equipment and lines. Similarly, in water treatment systems, paper and pulp processing pipelines, or disinfection systems, polymer scale can clog filters, membranes, pipelines, or nozzles, reducing efficiency and requiring frequent maintenance.

As mentioned above, the inventors have advantageously found that the presence of peroxy gluconic acid and/or percitric acid in the treatment fluid may prevent the formation of such polymer scale or may remove such polymer scale in various substrates or systems (e.g., the downhole environment). Accordingly, to address such polymer scale, the treatment fluids of embodiments of this disclosure containing peroxy gluconic acid and/or percitric acid may be introduced or placed into systems or substrates (e.g., the wellbore) using conventional techniques known in the art. If used as a preventative additive, the peroxy gluconic acid and/or percitric acid may be present in a treatment fluid used for oil and gas operations (e.g., as a drilling, completion, or workover fluid), water treatment, paper and pulp processing, or cleaning processes. If, however, remediation of a particular interval of the substrate (e.g., wellbore) is needed, peroxy gluconic acid and/or percitric acid may be present in a treatment fluid used as a remediation fluid which may be injected into such interval, in addition to other intervals. The treatment fluids above may contain an amount of peroxy gluconic acid and/or percitric acid sufficient to prevent polymer crosslinking, or alternatively to remediate polymer crosslinking. The treatment fluids may be used in conjunction with any process of water treatment, paper and pulp processing, cleaning, or oil and gas operations (e.g., drilling, completion, or production operation).

Thus, according to an embodiment, there is provided a method to prevent polymer scale (i.e. a polymer-polyvalent metal ion complex) from forming during an oil- and gas-field operation, water treatment, paper and pulp processing, or disinfection. The method includes introducing a treatment fluid containing peroxy gluconic acid and/or percitric acid into the system where polymer scale is present and allowing the treatment fluid to interact with the polymer scale for an effective amount of time to break down the scale and restore system performance. In oil- and gas-field operations, the method includes placing the treatment fluid containing peroxy gluconic acid and/or percitric acid into a wellbore in fluid communication with a subterranean formation where at least one of the wellbore or subterranean formation contains a polymer and a polyvalent metal ion and allowing the treatment fluid to interact with the polymer and polyvalent metal ion for an effective amount of time to prevent the polymer and polyvalent metal ion from complexing and forming polymer scale.

According to another embodiment, there is provided a method for removing polymer scale (i.e. a polymer-polyvalent metal ion complex) formed in a system or substrate during a process, such as water treatment, paper and pulp processing, disinfection activities, or oil- and gas-operations (e.g., wellbore or subterranean formation during an oil- and gas-field operation). The method includes introducing or placing a treatment fluid containing peroxy gluconic acid and/or percitric acid into the system or substrate in fluid communication with the polymer scale, where at least one component of the system or substrate contains polymer scale. The method further comprises allowing the treatment fluid to interact with or contact the polymer scale for an effective amount of time to remove the polymer scale from the system or substrate. In oil- and gas-operations, the method includes introducing or placing the treatment fluid containing peroxy gluconic acid and/or percitric acid into a wellbore in fluid communication with a subterranean formation, where at least one of the wellbore or subterranean formation contains polymer scale, and allowing the treatment fluid to interact with or contact the polymer scale for an effective amount of time to remove the polymer scale from the wellbore or subterranean formation.

According to another embodiment, the treatment fluid containing peroxy gluconic acid and/or percitric acid may be used in methods to remove polyvalent metal ions from water or process fluids (e.g., flowback fluids) to make such water or process fluids (e.g., flowback fluids) suitable for subsequent use in operations, such as oil- and gas-field operations, water treatment, paper and pulp processing, or disinfection activities. The method includes contacting the water or process fluid (e.g., flowback fluid) with the treatment fluid comprising peroxy gluconic acid and/or percitric acid for an effective amount of time to remove the polyvalent metal ions from the water or process fluid (e.g., flowback fluid) and thereby form a treated fluid.

A key benefit of removing or suppressing polyvalent metal ions from water or process fluids (e.g., flowback fluids) is that the treated fluid can be reused, for example, as a base fluid of a subsequent treatment fluid. In some cases, removal of polyvalent metal ions makes the treated fluid more suitable for eventual disposal.

Thus, in one embodiment, the method may further comprise using the treated fluid to form a new treatment fluid or disposing of the treated fluid. Optionally, the method may further comprise introducing the new treatment fluid into a system or substrate associated with industrial or operational processes, such as oil- and gas-field operations, water treatment, paper and pulp processing, or disinfection activities. Optionally, the method may further comprise introducing the new treatment fluid into a subterranean formation in oil- and gas-operations.

In one particular embodiment, there is provided a method to control iron in systems or substrates during industrial or operational processes, such as oil- and gas-field operations, water treatment, paper and pulp processing, or disinfection activities, using a treatment fluid containing peroxy gluconic acid and/or percitric acid. The method generally includes introducing the treatment fluid into the system or substrate containing iron and allowing the treatment fluid to interact with or contact the iron for an effective amount of time to control iron. For example, there is provided a method to control iron in subterranean formations during oil- and gas-field operations using the treatment fluid containing peroxy gluconic acid and/or percitric acid. The method generally includes injecting the treatment fluid into the subterranean formation containing iron and allowing the treatment fluid to interact with or contact the iron for an effective amount to time to control iron in the subterranean formation.

In one embodiment, the treatment fluid is substantially free of a chelating agent. Fe²⁺ is commonly forced into Fe³⁺ by traditional oxidizers or breakers (H₂O₂, Persulfates, Perborates, ClO₂); this effect is extremely detrimental to polymers commonly used, for example, in subterranean formations during oil- and gas-operations, as it only takes small amounts (up to about 25 ppm) of Fe³⁺ to truly cause negative effects (such effects being within 1 month of, for example, flocculation or crosslinking of the polymer). The surprising effect of peroxy gluconic acid alone or in combination with percitric acid according to the present disclosure is that it has the ability to bind to or otherwise absorb Fe²⁺ but more importantly Fe³⁺ in a rapid capacity, allowing its oxidizing capabilities to truly interact and successfully break polymers where traditional breakers will cause crosslinking and flocculation effects. Peroxy gluconic acid and/or gluconic acid may be more selective to binding with iron ions, whereas percitric acid and/or citric acid may bind more tightly with more ions but not selectively to iron ions.

The term “iron control” for the purpose of this application refers to the prevention or at least reduction of iron oxide precipitates (e.g., iron oxides) that form in a substrate or system (e.g., formation) during the treatment thereof as a consequence of iron cations that are naturally present in the substrate or system (e.g., formation) or iron cations that originate from the dissolution of iron from the equipment being used or other systems and processes (e.g., production of the well).

In oil and gas operations, iron control refers to preventing damage in formations and wellbores caused by ferric hydroxide solids or other insoluble iron salts. While the presence of iron cations may have other disadvantages when treating subterranean formations, the formation of ferric hydroxide solids is a substantial problem in formation treatments because in many cases it can lead to formation damage.

In water treatment, iron control involves minimizing fouling or scaling caused by iron oxides and iron sulfides in pipelines, membranes, and tanks. In paper and pulp processing, it prevents deposition of iron salts that can interfere with product quality and equipment efficiency. In disinfection systems, iron control reduces precipitates on sterilization equipment or surfaces, ensuring effective cleaning and minimizing microbial growth.

In some embodiments iron control may also refer to the prevention of asphaltic sludge, by preventing the polar groups in asphaltenes from reacting with the ferric iron. Iron control in other embodiments may also refer to the prevention of or at least reduction of the precipitation of any other type of insoluble iron salt, especially iron sulfides, which can form in environments containing H₂S or other contaminants (e.g. in the well and near-wellbore area especially in sour wells, i.e. wells containing H₂S).

Use of the above described treatment fluid containing peroxy gluconic acid and/or percitric acid to control iron may ensure that some chemical additives, such as chelating agents, corrosion inhibitors, anti-sludge agents, iron control agents, scale inhibitors, are needed in much smaller amounts (or may not be needed at all) to achieve a similar effect to that of state of the art treatment fluids. Accordingly, the chemicals burden of the method may be reduced creating a more sustainable way to produce oil and/or gas. Under some conditions these additives are even completely redundant, and in one embodiment the treatment fluid used for iron control is substantially free of a chelating agent.

Furthermore, in some embodiments, peroxy gluconic acid and/or percitric acid in combination with a biocide or bactericide in the treatment fluid may reduce the number of and sometimes even fully remove the bacteria responsible for the formation of sulfides from sulfate. As iron can form a precipitate with sulfide, in this way iron control also takes place. The combination of peroxy gluconic acid and/or percitric acid with a biocide or bactericide may be synergistic, i.e. less biocide or bactericide is required to control the growth of microorganisms in the presence of peroxy gluconic acid and/or percitric acid, reducing the negative environmental effect of using large quantities of biocides or bactericides with their inherent negative eco-tox profile.

Moreover, in another particular embodiment, there is provided a method for oxidizing a polymer while preventing a polymer-polyvalent metal ion complex deposit from forming during an industrial or operational process, such as oil- and gas-field operations, water treatment, paper and pulp processing, or disinfection activities. The method includes introducing the treatment fluid of the present disclosure into a system or substrate and allowing the treatment fluid to interact with the polymer for an effective period of time, where the system or substrate contains the polymer. For example, a method is provided for oxidizing a polymer while preventing a polymer-polyvalent metal ion complex deposit from forming during an oil- and gas-field operation by placing the treatment fluid of the present disclosure into a wellbore in fluid communication with a subterranean formation and allowing the treatment fluid to interact with the polymer for an effective period of time where at least one of the wellbore or the subterranean formation contains the polymer. As discussed above, the treatment fluid of the present disclosure has the ability to absorb Fe²⁺ and more importantly Fe³⁺ in a rapid capacity thus allowing the treatment fluid, and in particular, percitric acid, to oxidize the polymer more effectively than traditional oxidizing agents used in various processes (e.g., oil- and gas-field operations, water treatment, paper and pulp processing, or disinfection activities) for oxidizing the polymers above.

In yet another embodiment, the treatment fluid containing peroxy gluconic acid and/or percitric acid may be used in various applications, such as oil- and gas-field operations water treatment, paper and pulp processing, or disinfection, to at least: reduce microbial or viral populations on a surface or object or in liquid system containing a body or stream of water or process fluid (e.g., flowback fluid), or prevent biological fouling, or to control microorganism growth, or eliminate microbial contamination, or limit or prevent biological fouling in liquid systems containing water or process fluids (e.g., flowback fluids), or limit or prevent biological fouling on the surfaces of equipment that come in contact with such liquid systems. The method generally includes contacting the surface or object or liquid system with the treatment fluid containing peroxy gluconic acid and/or percitric acid for an effective amount of time to achieve the above-described effects.

Methods by which the treatment fluid comprising peroxy gluconic acid and/or percitric acid are contacted with the surface or object or introduced into the liquid system are not critical. Contacting with or introduction of the treatment fluid may be carried out in a continuous or intermittent manner and will depend on the type of surface or object or water or process fluid (e.g., flowback fluid) being treated. In one embodiment, the treatment fluid is added to water or flowback fluids to form a treated fluid in need of treatment prior to the drilling and fracking in order to restrict the introduction of microbes into the reservoir and to prevent the microbes from having a negative effect on the integrity of the fluids. The treated fluid may be used for both slick water fracturing, gel fracturing, water treatment systems, paper and pulp processing pipelines, disinfection equipment etc., depending on the type of process (e.g., formation being fractured) and its requirements (e.g., the type of hydrocarbon expected to be produced).

In a further embodiment, the treatment fluid comprising peroxy gluconic acid and/or percitric acid is added to water or process fluids (e.g., flowback fluids) in need of treatment before disposal. In such an embodiment, process fluids (e.g., flowback fluids from hydraulic fracturing or post-fracking, wastewater from water treatment systems, process water from paper and pulp processing) are treated to minimize microbial contaminations in the waters and to remove solids prior to disposal (e.g., into a subterranean well), reuse in a subsequent applications (e.g., fracturing application) or return into local environmental water sources.

According to embodiments, a sufficient amount of the treatment fluid containing peroxy gluconic acid and/or percitric acid is added to the water or process fluid (e.g., flowback fluid) in need of treatment to provide a desired peroxy gluconic acid and/or percitric acid concentration for antimicrobial efficacy. For example, water or process fluid (e.g., flowback fluid) is dosed until a peroxy gluconic acid and/or percitric acid concentration within the water of flowback fluid is detected within the preferred concentration range (for example, about 0.1 ppm to about 10,000 ppm or about 1 ppm to about 1000 ppm or about 1 ppm to about 100 ppm).

The above method requires a certain minimal contact time of the peroxy gluconic acid and/or percitric acid with the water or process fluid (e.g., flowback fluid) in need of treatment for occurrence of sufficient antimicrobial effect. In some embodiments, the contact time may be at least about 10 seconds. In some embodiments, the contact time may be at least about 1 minute or at least about 5 minutes or at least about 10 minutes, or at least about 30 minutes, or at least about 60 minutes.

According to another embodiment, the peroxy gluconic acid and/or percitric acid-containing compositions made by the methods of the present disclosure may be used to improve friction reducer hydration speeds in treatment fluids containing polymer friction reducers (e.g., acrylate, polyacrylamide, etc.). In particular, contacting of these treatment fluids with the composition containing peroxy gluconic acid and/or percitric acid may produce a treatment fluid that exhibits a viscosity that is higher than the viscosity of similar treatment fluids that have not been contacted by the composition containing peroxy gluconic acid and/or percitric acid for a certain of period time, such as from about 1 second or about 5 seconds or about 10 seconds or about 20 seconds or about 30 seconds after the treatment fluid has been contacted with the composition containing peroxy gluconic acid and/or percitric acid, to less than about 20 minutes, or less than about 15 minutes or less than about 10 minutes or less than about 5 minutes after the treatment fluid has been contacted with the composition containing peroxy gluconic acid and/or percitric acid depending on the specific process requirements. This is advantageous in applications where higher viscosity is needed, such as transporting fluids in pipelines, facilitating fiber suspension in paper processing, enabling effective friction reduction in water treatment systems, during crucial horizontal portions of a well completion etc.

The method generally includes contacting a treatment fluid comprising a polymer with the composition containing peroxy gluconic acid and/or percitric acid to form a treated treatment fluid. The treated fluid may then be used in various processes, such as hydraulic fracturing, membrane filtration, paper fiber transport, or cleaning and disinfection. For example, the method generally includes contacting a treatment fluid comprising a polymer with the composition containing peroxy gluconic acid and/or percitric acid to form a treated treatment fluid and transporting the treated treatment fluid into a wellbore in fluid communication with a subterranean formation wherein the treated treatment fluid exhibits a viscosity higher than the similar treatment fluid that has not been contacted by the composition containing peroxy gluconic acid and/or percitric acid for a period of time ranging from about 5 seconds after the treated treatment fluid has been formed to less than about 20 minutes, or less than about 15 minutes or less than about 10 minutes or 5 minutes after the treated treatment fluid has been formed.

The amount of peroxy gluconic acid present in the treated treatment fluid may be from about 0.1 ppm to about 70 wt. %, based on the total weight of the treated treatment fluid, or any range therein. For example, the amount of peroxy gluconic acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of peroxy gluconic acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treated treatment fluid.

The amount of percitric acid present in the treated treatment fluid may be from about 0.1 ppm to about 70 wt. %, based on the total weight of the treated treatment fluid, or any range therein. For example, the amount of percitric acid may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the amount of percitric acid may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treated treatment fluid.

The total amount of acid (peroxy gluconic acid and/or percitric acid) present in the treated treatment fluid may be from about 0.1 ppm to about 70 wt. %, based on the total weight of the treated treatment fluid, or any range therein. For example, the total amount of acid (peroxy gluconic acid and/or percitric acid) may range between about 0.25 ppm to about 50,000 ppm, or about 0.5 ppm to about 25,000 ppm, or about 0.75 ppm to about 10,000 ppm or about 1 ppm to about 5000 ppm or about 10 ppm to about 2500 ppm or about 25 ppm to about 1000 ppm, based on the total weight of the treatment fluid. In another embodiment, the total amount of acid (peroxy gluconic acid and/or percitric acid) may range from about 1 ppm to 1000 ppm, or from about 5 ppm to about 80 ppm, or from about 10 ppm to about 70 ppm, or from about 30 ppm to about 60 ppm, or from about 40 ppm to about 55 ppm, based on the total weight of the treated treatment fluid.

In still another embodiment, there is provided a method of enhancing the efficiency or productivity of an industrial or operational process by contacting a system, substrate, or environment with the treatment fluids of the present disclosure. This method is particularly advantageous when the treatment fluid is contacted with the system or substrate prior to a primary treatment or processing step. As an example, there is provided a method of enhancing productivity of hydrocarbons from a hydrocarbon bearing subterranean formation by contacting the formation with the treatment fluids of the present disclosure. The advantage of this method is especially achieved when such treatment fluid is contacted with the formation prior to hydraulic fracturing.

The method may generally include introducing a first treatment fluid comprising peroxy gluconic acid and/or percitric acid into the system or substrate at a location where treatment or stimulation is desired. The first treatment fluid is then allowed to interact with the system, substrate, or process fluid, and a second treatment fluid is subsequently introduced into the system or substrate to perform the primary treatment, such as enhancing fluid flow, cleaning surfaces, preventing fouling, or improving material processing efficiency. As an example, the method may generally include introducing a first treatment fluid comprising peroxy gluconic acid and/or percitric acid into the formation at a location where stimulation is desired. The first treatment fluid is then allowed to contact the formation and a second treatment fluid is subsequently injected into the subterranean formation at the location at a pressure sufficient to create or enlarge a fracture in the subterranean formation.

The amount of peroxy gluconic acid present in the first treatment fluid may be from about 0.01 wt. % to about 50 wt. %, based on the total weight of the treatment fluid, or any range therein. For example, the amount of peroxy gluconic acid may range between about 2 wt. % to about 50 wt. % or from about 5 wt. % to about 35 wt. % or from about 7 wt. % to about 25 wt. % or from about 10 wt. % to about 20 wt. %, based on the total weight of the treatment fluid. In another embodiment, the amount of peroxy gluconic acid present in the treatment fluid may be from about 0.01 wt. % to about 2 wt. % or from about 0.05 wt. % to about 1.75 wt. % or from about 0.1 wt. % to about 1.5 wt. % or from about 0.5 wt. % to about 1 wt. %, based on the total weight of the treatment fluid.

The amount of percitric acid present in the first treatment fluid may be from about 0.01 wt. % to about 50 wt. %, based on the total weight of the treatment fluid, or any range therein. For example, the amount of percitric acid may range between about 2 wt. % to about 50 wt. % or from about 5 wt. % to about 35 wt. % or from about 7 wt. % to about 25 wt. % or from about 10 wt. % to about 20 wt. %, based on the total weight of the treatment fluid. In another embodiment, the amount of percitric acid present in the treatment fluid may be from about 0.01 wt. % to about 2 wt. % or from about 0.05 wt. % to about 1.75 wt. % or from about 0.1 wt. % to about 1.5 wt. % or from about 0.5 wt. % to about 1 wt. %, based on the total weight of the treatment fluid.

The total amount of acid (peroxy gluconic acid and/or percitric acid) present in the first treatment fluid may be from about 0.01 wt. % to about 50 wt. %, based on the total weight of the treatment fluid, or any range therein. For example, the total amount of acid (peroxy gluconic acid and/or percitric acid) may range between about 2 wt. % to about 50 wt. % or from about 5 wt. % to about 35 wt. % or from about 7 wt. % to about 25 wt. % or from about 10 wt. % to about 20 wt. %, based on the total weight of the treatment fluid. In another embodiment, the total amount of acid (peroxy gluconic acid and/or percitric acid) present in the treatment fluid may be from about 0.01 wt. % to about 2 wt. % or from about 0.05 wt. % to about 1.75 wt. % or from about 0.1 wt. % to about 1.5 wt. % or from about 0.5 wt. % to about 1 wt. %, based on the total weight of the treatment fluid.

In yet another embodiment, there is provided a method of removing at least a portion of deposits from equipment (e.g., oilfield equipment) by injecting a treatment fluid containing peroxy gluconic acid and/or percitric acid into the equipment (e.g., oilfield equipment). The equipment may include all types and varieties of equipment associated with oil and gas recovery, water treatment, paper and pulp processing, disinfection systems, or processing or operations, such as, but not limited to, filters, membranes, casing, pumps, separators, tanks, lines, pipes, and the like. The method may be used with all such above equipment whether the equipment is positioned above or below the surface (e.g., downhole).

The injecting of the treatment fluid into the equipment (e.g., oilfield equipment) can normally be effected using methods known in the art, for example, by the use of pumps. In accordance with an embodiment, the treatment fluid can be kept in contact with the equipment (e.g., oilfield equipment) after injection for any period of time, such as a period of time ranging from about 1 second to about 24 hours. The injecting may be effected in a single step or in more than one step. In some embodiments, after an initial injection, the treatment fluid may be circulated inside the equipment (e.g., oilfield equipment) by the use of pumps for any period of time, such as from about 1 minute to about 24 hours.

Deposits removed from the equipment (e.g., oilfield equipment) by the present method may include deposits formed of organic material, inorganic material, or mixtures thereof, such as hydrocarbons, scale, contaminants, or a mixture thereof (specific to the application). Hydrocarbons include, but are not limited to, crude oil, shale oil, tar, asphaltene, and paraffin. Scale includes, but is not limited to, any carbonate, sulfate, or metal salt that has precipitated out of the water phase, trace formation material such as silica and clays, and corrosion materials. In water treatment systems, deposits may include biofilms, scale, or fouling residues. In paper and pulp processing, contaminants may include cellulose fibers, polymeric residues, or mineral deposits. In disinfection systems, deposits may include organic films, biofilms, or residues from cleaning agents. In certain embodiments, after the treatment fluid has been injected into and contacted with the equipment (e.g., oilfield equipment), the equipment (e.g., oilfield equipment) is substantially free of such deposits.

Although the above method was directed to the removal of deposits from oilfield equipment, the method may be used to remove any type of contaminant material (for e.g. organic material, inorganic material or mixtures thereof) from any type of surface, including both metallic and non-metallic surfaces, of one or more pieces of industrial equipment. The method includes contacting the surface having a contaminant thereon with a composition comprising peroxy gluconic acid and/or percitric acid according to this disclosure for an effective period of time such that at least a portion of the contaminant is removed from the surface. The composition may optionally include one or more of a corrosion inhibitor and chemical additive described above.

Metallic surfaces may include ferrous and non-ferrous surfaces. Ferrous surfaces include, but are not limited to, steel, stainless steel, cold-rolled steel, cast iron, tin-plated steels, copper-plated steels, organic-coated steels, galvanized steels and zinc/aluminum galvanized steels. Non-ferrous surfaces include, but are not limited to, aluminum and aluminum alloys, zinc and zinc-based alloys, zinc-aluminum alloys, and copper and copper.

Non-metallic surfaces include plastics, including but not limited to, polycarbonates, polyvinyl chlorides, polyethylenes, polypropylenes, thermoplastic polyesters or polyamides, polyurethanes, epoxides or polyepoxies, polystyrene or its copolymers, nylons and modified polyamides, and modified celluloses.

Without limitation, the composition containing peroxy gluconic acid and/or percitric acid may disaggregate and/or dissolve contaminant materials from surfaces of industrial equipment within industrial facilities (for e.g., oil refineries, natural gas processing plants, petrochemical facilities, port terminals, chemical plants, industrial and institutional (I & I) settings, such as with dairy and food processing plant, and the like). In embodiments, the composition containing peroxy gluconic acid and/or percitric acid may be used to remove a contaminant material from the surface of any piece of industrial equipment used in industrial facilities including vessels, tanks, vacuum towers, heat exchangers, piping, distillation columns, and the like. In embodiments, contaminant materials to be removed may include any contaminant material produced, stored, transported, or the like during the process of crude oil refinement, natural gas processing, hydrocarbon transport, hydrocarbon processing, hydrocarbon cleanup, chemical processing, food and dairy processing and the like. In embodiments, examples of contaminant materials may include, but are not limited to, residual oil, hydrogen sulfide, combustible gas, iron sulfides, the like, or any combinations thereof. In embodiments, the contaminant materials are contacted with the composition containing peroxy gluconic acid and/or percitric acid, such that the contaminant materials are disaggregated and/or dissolved and may then be subsequently removed from the surface of the industrial equipment. The contaminant materials may be oxidized in the method and reduced to a harmless form.

As discussed above, the contaminant material removal method may include contacting the contaminant materials and/or the surface of the industrial equipment with the composition containing peroxy gluconic acid and/or percitric acid. For example, in embodiments comprising a vessel containing contaminant materials disposed within, the composition may be poured, pumped, injected, or using any other suitable means, into the vessel such that the composition contacts the contaminant materials disposed therein. As another example, in embodiments comprising industrial equipment having contaminant materials disposed thereon, the composition containing peroxy gluconic acid and/or percitric acid may be poured onto the contaminated portion of the industrial equipment or the contaminated portion of the industrial equipment may be submerged in the composition containing peroxy gluconic acid and/or percitric acid such that the composition contacts the contaminant materials. In another embodiment, the composition containing peroxy gluconic acid and/or percitric acid may be circulated though the contaminated equipment.

Any suitable timeframe during which the composition containing peroxy gluconic acid and/or percitric acid is in contact with the contaminant materials may be used as an effective period of time. In embodiments, the timeframe may extend for a sufficient period whereby at least a portion of the contaminant materials are removed (i.e., disaggregated, dissolved, emulsified, neutralized, and/or oxidized). In an embodiment, the timeframe may be from about 30 seconds to about one day, such as at least about 1 minute or at least about 5 minutes, or at least 15 about minutes, or at least about 30 minutes or at least about 1 hour, or at least 1.5 about hours or at least about 2 hours. In alternative embodiments, the time frame may be from about 1 minute to about twelve hours. In further alternative embodiments, the time frame may be from about one minute to about six hours.

In embodiments, once at least a portion of the contaminant materials have been removed, the contaminant materials may reside in the composition containing peroxy gluconic acid and/or percitric acid and may therefore be fluid and/or flowable within the composition. The contaminant materials residing within the composition may then be pumped, poured, or otherwise removed from the industrial equipment along with the composition.

The following clauses illustrated example subject matter described herein.
Clause 1. A method of producing a treatment fluid, comprising: contacting gluconic acid and hydrogen peroxide to form a composition comprising peroxy gluconic acid; and adding one or more chemical additives to the composition, wherein the peroxy gluconic acid is present in an amount greater than 10 wt. %, based on the total weight of the treatment fluid.
Clause 2. The method of Clause 1, wherein contacting gluconic acid and hydrogen peroxide to form a composition comprising peroxy gluconic acid comprises contacting gluconic acid, citric acid, and hydrogen peroxide to form a composition comprising peroxy gluconic acid and percitric acid.
Clause 3. The method of Clause 1 or Clause 2, wherein the one or more chemical additives comprises a corrosion inhibitor, wherein the corrosion inhibitor comprises an alkali metal polyphosphate having a general formula Mn+2PnO3n+1 where M is sodium, potassium or ammonium and n is an integer of at least 2.
Clause 4. The method of any one of Clauses 1 through 3, wherein the treatment fluid has a shelf-life stability configured to retain at least about 80% of the peroxy gluconic acid activity after at least 1 month at a temperature ranging from about 20° C. to about 70° C.
Clause 5. The method of any one of Clauses 1 through 4, wherein the one or more chemical additives comprises at least one of a corrosion inhibitor, a polymer, a wetting agent, an organophilic clay, an oxygen scavenger, a proppant, a biocide, a weighting agent, a foaming agent, a surfactant, a dispersant, an interfacial tension reducer, a solvent, a thinning agent, a stabilizing agent/pH buffer, and a scale inhibitor.
Clause 6. The method of any one of Clauses 1 through 5, wherein contacting gluconic acid and hydrogen peroxide to form a composition comprising peroxy gluconic acid comprises contacting gluconic acid, citric acid, and hydrogen peroxide to form a composition comprising peroxy gluconic acid and percitric acid, wherein the treatment fluid is substantially free of peracetic acid and performic acid.
Clause 7. A method for degrading a polymer from a subterranean formation in fluid communication with a wellbore, wherein the method comprises: contacting gluconic acid and hydrogen peroxide to form a composition comprising peroxy gluconic acid; adding one more chemical additives to the composition to define a treatment fluid, wherein the peroxy gluconic acid is present in an amount greater than 10 wt. %, based on the total weight of the treatment fluid; injecting the treatment fluid into the wellbore; and contacting the polymer with the treatment fluid for an effective amount time to degrade the polymer.
Clause 8. The method of Clause 7, wherein at least one of the wellbore and the subterranean formation contains a polymer scale, wherein contacting the polymer comprises contacting the polymer scale with the treatment fluid for an effective amount of time to remove the polymer scale from the at least one of the wellbore and the subterranean formation.
Clause 9. The method of Clause 7 or Clause 8, wherein the treatment fluid comprises a first treatment fluid, wherein the method further comprises: injecting a second treatment fluid comprising a polymer into the subterranean formation; and breaking the viscosity of the second treatment fluid by fragmenting the polymer to form a reduced viscosity treated fluid, wherein the polymer is fragmented by injecting the first treatment fluid into the subterranean formation.
Clause 10. The method of any one of Clauses 7 through 9, wherein the reduced viscosity treatment fluid is recovered from the subterranean formation.
Clause 11. The method of any one of Clauses 7 through 10, wherein the first treatment fluid further comprises a friction reducer, wherein the method further comprises contacting the second treatment fluid with the first treatment fluid to form a mixture, wherein a viscosity of the mixture of the first treatment fluid and the second treated treatment fluid is greater than a viscosity of the second treatment fluid for a period of time ranging from about 1 second after contacting to less than about 20 minutes after contacting.
Clause 12. The method of any one of Clauses 7 through 11, wherein the method further comprises injecting the treatment fluid into a component of a fluid handling system in fluid communication with the wellbore; and contacting the component of the fluid handling system with the treatment fluid for an effective amount to time to at least one of fragment the polymer on or within the component and remove a contaminate from a surface of the component.
Clause 13. The method of any one of Clauses 7 through 12, wherein the contacting of the component comprises flowing the treatment fluid through the component of the fluid handling system.
Clause 14. The method of any one of Clauses 7 through 13, further comprising washing or flushing the component of the fluid handling system with water or a flowback fluid to remove the fragmented polymer from the component.
Clause 15. The method of any one of Clauses 7 through 14, further comprising contacting at least one of the wellbore and the subterranean formation with the treatment fluid for an effective amount of time to reduce at least one of a microbial population and a viral population.
Clause 16. A method for preventing a polymer-polyvalent metal ion complex deposit from forming during an oil- and gas-field operation, wherein the method comprises: injecting a treatment fluid into a wellbore in fluid communication with a subterranean formation, wherein the treatment fluid comprises gluconic acid and hydrogen peroxide forming a composition comprising peroxy gluconic acid present in an amount greater than 10 wt. %, based on the total weight of the treatment fluid and one more chemical additives, wherein at least one of the wellbore or the subterranean formation contains a polymer and the polyvalent metal ion; and allowing the treatment fluid to interact with the polymer and the polyvalent metal ion for an effective amount of time to prevent the polymer and polyvalent metal ion from complexing and depositing within at least one of the wellbore and the subterranean formation.
Clause 17. The method of Clause 16, wherein the polyvalent metal comprises Fe²⁺, Fe³⁺o r a mixture thereof.
Clause 18. The method of Clause 16 or Clause 17, wherein the method further comprises contacting water or flowback fluid with the treatment fluid for an effective amount of time to remove or suppress the polyvalent metal ions from the water or flowback fluid and thereby form a treated fluid.
Clause 19. The method of any one of Clauses 16 through 18, wherein the treatment fluid comprises gluconic acid, citric acid, and hydrogen peroxide forming a composition comprising peroxy gluconic acid and percitric acid present in an amount greater than 10 wt. %, based on the total weight of the treatment fluid.
Clause 20. The method of any one of Clauses 16 through 18, wherein the treatment fluid is substantially free of chelating agents other than gluconic acid, peroxy gluconic acid, citric acid, or percitric acid.
Clause 21. A method for treating a wellbore or subterranean formation, comprising: providing a treatment fluid comprising gluconic acid and hydrogen peroxide; and contacting at least one of the wellbore and the subterranean formation with the treatment fluid for an effective amount of time to at least one of remove a contaminant, reduce a microbial population, or prevent formation of a polymer-polyvalent metal ion complex deposit.
Clause 22. The method of Clause 21, wherein the treatment fluid further comprises citric acid.
Clause 23. The method of Clause 21 or Clause 22, wherein the treatment fluid comprises from about 1 ppm to about 40 wt % of the gluconic acid and hydrogen peroxide combined, based on a total weight of the treatment fluid.
Clause 24. The method of any one of Clauses 21 through 23, wherein the treatment fluid further comprises methanol in an amount of about 15 wt % to about 30 wt %, based on a total weight of the treatment fluid.
Clause 25. The method of any one of Clauses 21 through 24, wherein the treatment fluid has a freeze point of about 0° F. to about −40° F.
Clause 26. The method of any one of Clauses 21 through 25, wherein the contaminant comprises a polymer-polyvalent metal ion complex.
Clause 27. The method of any one of Clauses 21 through 26, wherein the polyvalent metal ion comprises iron.
Clause 28. The method of any one of Clauses 21 through 27, further comprising flowing the treatment fluid through a component of a fluid handling system to remove a contaminant from a surface of the component.
Clause 29. The method of any one of Clauses 21 through 28, further comprising flowing the treatment fluid through a component of a fluid handling system to remove a contaminant from a surface of the component and washing or flushing the component with water or a flowback fluid to remove the contaminant from the component.
Clause 30. A treatment fluid for use in oil- and gas-field operations, comprising: gluconic acid; hydrogen peroxide; and optionally citric acid, wherein the treatment fluid is configured to prevent or remove polymer-polyvalent metal ion complex deposits in at least one of a wellbore and a subterranean formation.
Clause 31. The treatment fluid of Clause 30, wherein the treatment fluid comprises from about 1 ppm to about 40 wt % of the gluconic acid and hydrogen peroxide combined, based on a total weight of the treatment fluid.
Clause 32. The treatment fluid of Clause 30 or Clause 31, further comprising methanol in an amount of about 15 wt % to about 30 wt %, based on a total weight of the treatment fluid.
Clause 33. The treatment fluid of any one of Clauses 30 through 32, wherein the treatment fluid has a freeze point of about 0° F. to about −40° F.
Clause 34. The treatment fluid of any one of Clauses 30 through 33, further comprising citric acid, wherein the gluconic acid is present in a greater amount than the citric acid.
Clause 35. The treatment fluid of any one of Clauses 30 through 34, wherein the treatment fluid is substantially free of a chelating agent other than the gluconic acid and citric acid.
Clause 36. The treatment fluid of any one of Clauses 30 through 35, wherein the polymer-polyvalent metal ion complex comprises iron.
Clause 37. A method of formulating a treatment fluid for use in oil- and gas-field operations, comprising: combining gluconic acid and hydrogen peroxide to form a treatment fluid; and optionally adding citric acid to the treatment fluid, wherein the treatment fluid is configured to selectively bind to iron ions over calcium ions when used in at least one of a wellbore and a subterranean formation.
Clause 38. The method of Clause 37, further comprising adding methanol to the treatment fluid in an amount of about 15 wt % to about 38 wt %, based on a total weight of the treatment fluid, to achieve a freeze point of about 0° F. to about −40° F.
Clause 39. The method of Clause 37 or Clause 38, wherein the gluconic acid is present in a greater amount than the citric acid.
Clause 40. The method of any one of Clauses 37 through 39, wherein the treatment fluid comprises from about 1 ppm to about 40 wt % of the gluconic acid and hydrogen peroxide combined, based on a total weight of the treatment fluid.

What is claimed is:

1. A method for treating a substrate, comprising:
providing a treatment fluid comprising an oxidation product of glucose and hydrogen peroxide; and
contacting the substrate with the treatment fluid for an effective amount of time to at least one of remove a contaminant, reduce a microbial population, or prevent formation of a polymer-polyvalent metal ion complex deposit.

2. The method of claim 1, wherein the oxidation product of glucose comprises gluconic acid and glucaric acid, and wherein the treatment fluid comprises from about 1 ppm to about 40 wt % of the oxidation product of glucose and hydrogen peroxide combined, based on a total weight of the treatment fluid.

3. The method of claim 1, wherein the treatment fluid further comprises citric acid.

4. The method of claim 1, wherein the treatment fluid further comprises methanol in an amount of about 15 wt % to about 30 wt %, based on a total weight of the treatment fluid.

5. The method of claim 1, wherein the treatment fluid has a freeze point of about 0° F. to about −40° F.

6. The method of claim 1, wherein the contaminant comprises a polymer-polyvalent metal ion complex.

7. The method of claim 1, wherein the polymer-polyvalent metal ion comprises iron.

8. The method of claim 1, further comprising flowing the treatment fluid through a component of a fluid handling system to remove a contaminant from a surface of the component.

9. The method of claim 1, further comprising flowing the treatment fluid through a component of a fluid handling system to remove a contaminant from a surface of the component and washing or flushing the component with water or a flowback fluid to remove the contaminant from the component.

10. A treatment fluid, comprising:
gluconic acid;
glucaric acid; and
hydrogen peroxide;
wherein the treatment fluid is configured to prevent or remove polymer-polyvalent metal ion complex deposits in a substrate.

11. The treatment fluid of claim 10, wherein the treatment fluid comprises from about 1 ppm to about 40 wt % of the gluconic acid and hydrogen peroxide combined, based on a total weight of the treatment fluid.

12. The treatment fluid of claim 10, further comprising methanol in an amount of about 15 wt % to about 30 wt %, based on a total weight of the treatment fluid.

13. The treatment fluid of claim 10, wherein the treatment fluid has a freeze point of about 0° F. to about −40° F.

14. The treatment fluid of claim 10, further comprising citric acid, wherein the gluconic acid is present in a greater amount than the citric acid.

15. The treatment fluid of claim 10, wherein the treatment fluid is substantially free of a chelating agent other than the gluconic acid, the glucaric acid, and, optionally, citric acid.

16. The treatment fluid of claim 10, wherein the polymer-polyvalent metal ion complex comprises iron.

17. A method of formulating a treatment fluid, comprising:
combining gluconic acid and hydrogen peroxide to form a treatment fluid; and
adding citric acid to the treatment fluid,
wherein the treatment fluid is configured to selectively bind to iron ions over calcium ions.

18. The method of claim 17, further comprising adding methanol to the treatment fluid in an amount of about 15 wt % to about 38 wt %, based on a total weight of the treatment fluid, to achieve a freeze point of about 0° F. to about −40° F.

19. The method of claim 17, wherein the gluconic acid is present in a greater amount than the citric acid.

20. The method of claim 17, wherein the treatment fluid comprises from about 1 ppm to about 40 wt % of the gluconic acid and hydrogen peroxide combined, based on a total weight of the treatment fluid.
